(19) United States
(12) Patent Application Publication
(10) Pub. No.: US 2026/0069664 A1
(43) Pub. Date: Mar. 12, 2026

(54) COMPOSITIONS AND METHODS FOR TREATING VENOUS BLOOD CLOTS

(71) Applicants: The University of North Carolina at Chapel Hill, Chapel Hill, NC (US); NEW YORK UNIVERSITY, New York City, NY (US)

(72) Inventors: Edward Suarez Moreira Bahnson, Chapel Hill, NC (US); Sophie Licia Maiocchi, Durham, NC (US); Nathalie May Pinkerton, New York, NY (US)

(21) Appl. No.: 19/105,512

(22) PCT Filed: Aug. 30, 2023
(86) PCT No.: PCT/US2023/031542

§ 371 Date: Feb. 21, 2025

Related U.S. Application Data
(60) Provisional application No. 63/402,758, filed on Aug. 31, 2022.

(51) Int. Cl. A61K 38/46; A61K 9/50; A61K 9/51; A61K 35/15; A61P 7/02

(52) U.S. Cl. CPC A61K 38/465; A61K 9/5073; A61K 9/51; A61K 9/5123; A61K 9/5146; A61K 9/5169; A61K 35/15; A61P 7/02; C12Y 301/21001

(57) ABSTRACT

Therapeutic compositions for treating a subject, the compositions including a polymeric nanoparticle, an active agent encapsulated within the polymeric nanoparticle, and a delivery vehicle for targeted delivery of the polymeric nanoparticle and encapsulated agent to a target tissue in the subject. The therapeutic compositions can be designed for targeting the destruction of a Nuclei neutrophil extracellular trap (NET) in a venous thrombus in a subject. The active agent can be an enzyme, including deoxyribonuclease 1 (DNase 1) enzyme. Methods of treating a subject using the therapeutic compositions are provided, including methods of treating venous thrombus, deep vein thrombosis, or a related blood clotting disease and/or condition.

### PRIORITY CLAIM

The present application claims the benefit of U.S. Provisional Patent Application Ser. No. 63/402,758, filed Aug. 31, 2022, the disclosure of which is incorporated herein by reference in its entirety.

### GRANT STATEMENT

This invention was made with government support under Grant Number HL145354 awarded by the National Institutes of Health. The government has certain rights in the invention.

### TECHNICAL FIELD

The subject matter disclosed herein relates generally to compositions and methods for treating venous blood clots. More particularly, the subject matter disclosed herein relates to compositions for treating a subject, the compositions including a polymeric nanoparticle, an active agent encapsulated within the polymeric nanoparticle, and a delivery vehicle for targeted delivery of the polymeric nanoparticle and encapsulated agent to a target tissue in the subject.

### BACKGROUND AND INTRODUCTION

Approximately 900,000 Americans are affected by deep vein thrombosis (DVT) each year with 10-30% of these patients dying within 1 month of diagnosis¹, and 20-50% suffering in the long-term from disabling post-thrombotic syndrome (PTS)². Additionally, DVT and pulmonary embolism (PE) have emerged as a frequent and life-threatening complication in critically ill COVID-19 patients³. Oral anticoagulants are currently the standard treatment for DVT; however, this only prevents further clotting and does not remove the existing thrombus. Rather, the goal in the treatment of thrombosis should be the restoration of adequate blood flow by lysing the occluding thrombus either by i) lysis of the fibrin meshwork of thrombi with fibrinolytic enzymes (e.g. tissue plasminogen activator, tPA), ii) mechanical clot removal, or iii) catheter-directed fibrinolytic delivery. Unfortunately, one of the key issues facing current thrombolytic therapy is that as thrombi age, the thrombolytics such as tPA do not penetrate the thrombus deeply and only act on the thrombus surface^{4, 5}. Moreover, both systemic thrombolytic therapy and catheter directed thrombolytic injection are accompanied by unwanted high-risk side effects, such as intracranial bleeding. Thus, there remains an unmet need for targeted delivery of anti-thrombotic therapeutics to treat DVT and related conditions, and a need to explore alternative therapeutic targets that do not promote bleeding. These needs are addressed by this disclosure.

### SUMMARY

This summary lists several embodiments of the presently disclosed subject matter, and in many cases lists variations and permutations of these embodiments. This summary is merely an example of the numerous and varied embodiments. Mention of one or more representative features of a given embodiment is likewise for purposes of example. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features. In some embodiments, provided are therapeutic compositions for treating a subject, the therapeutic compositions comprising a nanoparticle; an active agent, wherein the active agent is encapsulated within the nanoparticle; and a delivery vehicle for targeted delivery of the nanoparticle and encapsulated agent to a target tissue in the subject. In some embodiments, the active agent is releasable from the delivery vehicle at the target tissue. In some embodiments, the active agent comprises a small molecule, nucleic acid, therapeutic protein and/or an active enzyme. In some embodiments, the active agent comprises Deoxyribonuclease 1 (DNase 1), optionally wherein the active agent comprises a nucleic acid encoding for DNase 1.

In some embodiments, he delivery vehicle is a functionalized delivery vehicle, wherein the functionalized delivery vehicle comprises a cell or cellular component for cell-mediated delivery of the nanoparticle. In some embodiments, the cell or cellular component for cell-mediated delivery of the nanoparticle comprises an immune cell. In some embodiments, the cell or cellular component for cell-mediated delivery of the nanoparticle comprises a macrophage or neutrophil. In some embodiments, the cell or cellular component for cell-mediated delivery of the nanoparticle comprises any autologous cell.

In some embodiments, the functionalized delivery vehicle for targeted delivery of the nanoparticle to a target tissue in the subject comprises a delivery vehicle decorated with ligands with an affinity for immune cells. In some embodiments, the functionalized delivery vehicle for targeted delivery of the nanoparticle to a target tissue in the subject comprises a delivery vehicle decorated with ligands with an affinity for a macrophage or neutrophil, optionally any autologous cell. In some embodiments, the active agent maintains activity upon release from the delivery vehicle at the target tissue. In some embodiments, the active agent maintains activity of at least about 50%, about 60%, about 70%, about 80%, about 90%, about 95%, about 99% or more of an activity of the active agent prior to release from the delivery vehicle. In some embodiments, the active agent comprises an enzyme with maintained enzymatic activity upon release at the target tissue. In some embodiments, the enzyme maintains activity of at least about 50%, about 60%, about 70%, about 80%, about 90%, about 95%, about 99% or more of an activity of the enzyme prior to release from the delivery vehicle.

In some embodiments, the nanoparticle is a polymeric nanoparticle, wherein the nanoparticle is surrounded by a protective layer of polymers. In some embodiments, the protective layer of polymers comprises a plurality of layers of polymers, optionally at least one, two or three layers of polymers. In some embodiments, at least one of the plurality of layers of polymers is substituted for a layer of protein. In some embodiments, the layer of protein comprises albumin. In some embodiments, the layer of protein stabilizes the nanoparticle. In some embodiments, the protective layer of polymers comprises one or more layers of polyethylene glycol (PEG). In some embodiments, the nanoparticle comprises a poly-acrylic acid, complexed with spermine and/or triethylamine, and coated one or more layers of PEG. In some embodiments, the nanoparticle comprises dual encapsulated active agents, optionally two separate active agents comprising a biologic and a small molecule. In some embodiments, the polymeric nanoparticle for encapsulation of an active agent is made by inverse Flash Nanopreciptation (iFNP) with stabilization of the nanoparticle using a polymer, an aqueous protein, or a combination thereof. In some embodiments, the composition further comprises CaCl2) to aid in the maintance of DNAse 1 activity upon release.

In some aspects, provided herein is a therapeutic composition for targeting the destruction of a neutrophil extracellular trap (NET) in a venous thrombus in a subject, the therapeutic composition comprising a nanoparticle encapsulating a Deoxyribonuclease 1 (DNase 1) enzyme, wherein the DNase 1 is releasable from the nanoparticle at the venous thrombus, wherein the DNase 1 maintains activity upon release. In some embodiments, the therapeutic composition comprises CaCl2) to aid in the maintenance of DNAse 1 activity upon release. In some embodiments, the nanoparticle comprises a layer of polyethylene glycol (PEG) and/or a layer of albumin. In some embodiments, the therapeutic composition comprises a cell or cellular component for cell-mediated delivery of the nanoparticle to a venous thrombus in the subject. In some embodiments, the cell or cellular component for cell-mediated delivery of the nanoparticle comprises an immune cell. In some embodiments, the cell or cellular component for cell-mediated delivery of the nanoparticle comprises a macrophage or neutrophil. In some embodiments, the cell or cellular component for cell-mediated delivery of the nanoparticle comprises any autologous cell. In some embodiments, the functionalized delivery vehicle for targeted delivery of the nanoparticle to a target tissue in the subject comprises a delivery vehicle decorated with ligands with an affinity for immune cells. In some embodiments, the functionalized delivery vehicle for targeted delivery of the nanoparticle to a target tissue in the subject comprises a delivery vehicle decorated with ligands with an affinity for a macrophage or neutrophil, optionally any autologous cell.

Provided herein in some aspects are methods of treating a condition in a subject in need of treatment, the methods comprising administering to a subject a therapeutic composition as disclosed herein, whereby the subject's condition is treated. In some embodiments, the subject is suffering from an inflammatory condition, wherein the inflammatory condition recruits neutrophils and/or macrophages, whereby administration of the therapeutic composition comprising a functionalized delivery vehicle or cell carrier targets the therapeutic composition to the site of the inflammatory condition. In some embodiments, the subject is suffering from venous thrombus, deep vein thrombosis, or a related blood clotting disease and/or condition. In some embodiments, administration of the therapeutic composition targets destruction of a neutrophil extracellular trap (NET) in a venous thrombus of the subject, optionally wherein intra-thrombus delivery of a thrombolytic enzyme, e.g. DNase 1, confers thrombolysis of venous thrombi in the subject without impacting surrounding tissues. In some embodiments, the therapeutic composition confers intra-thrombus delivery of a thrombolytic enzyme to a hardened thrombus with greater penetration of the thrombus as compared to other treatments, whereby the therapeutic composition confers greater thrombolysis of a thrombus with fewer side-effects.

In some aspects, provided are methods of making a polymeric nanoparticle for encapsulation of an active agent, the method comprising inverse Flash Nanopreciptation (iFNP) with stabilization of the nanoparticle with a polymer, an aqueous protein instead of a polymer, or a combination thereof. In some embodiments, such methods further comprise using dioxane, e.g. 1,4-dioxane, and/or CaCl2), to maintain activity of the active agent.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described herein below.

### BRIEF DESCRIPTION OF THE DRAWINGS

The features and advantages of the present subject matter will be more readily understood from the following detailed description which should be read in conjunction with the accompanying drawings that are given merely by way of explanatory and non-limiting example, and in which:

FIGS. 1A and 1B show the formation of inverse nanocarrier of DNase 1. FIG. 1A depicts normalized intensity distribution size measurement via dynamic light scattering of the inverse nanocarrier of DNase 1. FIG. 1B depicts the correlation function of DNase 1 with (solid blue line; also labeled with indicating arrow) and without (solid red line; also labeled with indicating arrow) the addition of a stabilizing block copolymer: Poly(D,L-lactide-block-acrylic acid (PLA-PAA, 9k-9k).

FIGS. 2A-2C show that spermine stabilizes the inverse nanocarrier by ionic complexation. FIG. 2A depicts the correlation function of the inverse nanocarrier diluted 10-fold into 1,4-dioxane solvent with (solid blue line; also labeled with indicating arrow) and without (solid red line; also labeled with indicating arrow) the addition of spermine (10 mM) in the chloroform quench bath. FIG. 2B depicts normalized intensity distribution size measurement via dynamic light scattering of the inverse nanocarrier of DNase 1 (black dashed line), followed by the processing steps of extraction (solid red line; also labeled with indicating arrow) and solvent swap (solid green line; also labeled with indicating arrow).

FIGS. 3A and 3B show the results of the formation of the PEG-coated DNase 1 nanoparticle. FIG. 3A depicts the normalized intensity distribution size measurement via dynamic light scattering of the inverse nanocarrier of DNase 1 (black dashed line) compared to the PEG-coated DNase 1 nanoparticle (blue solid line). FIG. 3B shows the normalized intensity distribution size measurement via dynamic light scattering of the PEG-coated DNase 1 nanoparticle (black dashed line), compared to the same nanoparticle but including the addition of 1 wt % of rubrene, relative to the amount of PEG block copolymer added (red solid line). FIG. 3C includes images of Transmission Electron Microscopy (TEM) of Rubrene-containing DNAse-1-nanoparticles. Particles were adsorbed onto copper 400 mesh TEM grids were negatively stained with 2% uranyl acetate and imaged via TEM at 100,000× and 200,0000× magnification (scale bar=200 nm).

FIGS. 4A and 4B show the results of detection of DNase 1 protein and recovered activity in PEG-coated DNase 1 nanoparticles. FIG. 4A depicts protein detection of DNase 1 via silver stain. Undiluted DNase 1 nanoparticles and DNase 1 standards were boiled with SDS loading buffer and then underwent gel electrophoresis followed by staining of the gel with Silver Stain Plus Kit (Biorad). Gels were imaged and band intensities quantified with Image J. DNase 1 protein content in the nanoparticles was quantified via extrapolating from a linear regression of the DNase 1 protein standards. This figure represents 2 independent batches of PEG-coated DNase 1 nanoparticles denoted by their IDs: ID22_14 and ID22_15. FIG. 4B depicts the detection of recovered DNase 1 activity in PEG-coated DNase 1 nanoparticles via Denatured Gel Zymography Assay. DNase 1 nanoparticles were degraded by stirring in 5% SDS, 0.1M NaOH for 48 hrs. This sample was then diluted and boiled in SDS sample buffer before undergoing gel electrophoresis in a denatured DNA-containing gel. DNase 1 can then be refolded by incubation with SDS-free refolding buffers and DNA detected by staining with 1×Sybr Safe DNA stain (Thermo Fisher).

FIGS. 5A-5C show the drug release curve from the DNAse 1 nanoparticles. The release curve show a burst release in the first 3 hours followed by sustained slow release for 48 h. FIG. B and 5C show the degradation of Neutrophil Extracellular Traps (NETs) by DNase 1 nanoparticles. FIG. 5B shows representative fluorescence images of Sytox-green stained NETs. NETs were generated by stimulating murine bone marrow derived neutrophils with 100 nM PMA for 4 hours at 37° C. with 5% CO2 and humidity. These NETs were then incubated with 10 mM Tris-HCl buffer containing 3 mM CaCl₂ and 3 mM MgCl₂ for up to 6 hrs in the absence or presence of DNase 1 alone (1 U/mL) or DNase 1-nanoparticles (1 U/mL). After stopping the reaction with 2 mM EDTA and fixing the NETs with 2% PFA/1×PBS, the DNA was stained with Sytox Green and imaged fluorescently. Left-most upper panel: NETs degraded by DNase 1 alone (1 U/mL, 10 min). Left-most lower panel: Sytox-green staining produced by un-stimulated neutrophils. Middle upper and lower panel: NETs incubated for up to 6 hrs in the presence (upper) or absence (lower) of DNase1-NPs (1 U/mL). Right-most upper and lower panel: NETs incubated for up to 4 hrs in the presence (upper) or absence (lower) of DNase1-NPs (1 U/mL). FIG. 5C shows quantification of NETs by Area coverage of NETs following treatment with DNase 1 alone or DNase 1 nanoparticles. 3-4 independent biological replicates are shown with 4-8 technical replicates per experiment. Samples taken at 1, 2, 4 and 6 hrs (left to right, respectively, on the graph) Data is presented as the mean±the S.E.M. Data was analyzed via factorial ANOVA of the log transformed ratio of area in DNase1-NP treated conditions vs area in time-matched untreated conditions. ***=P<0.001, ****=P<0.0001.

FIGS. 6A-6D show the results of internalization of rubrene-DNase 1-nanoparticles by murine bone marrow derived neutrophils. Rubrene-DNase 1 nanoparticles were incubated for up to 1.5 hr with murine bone marrow derived neutrophils with a final concentration of ˜0.2 μg/mL of rubrene at either 4° C. or 37° C. Neutrophils were then labelled with live/dead violet stain and CD45 antibody. FIG. 6A shows confocal fluorescence microscopy images of murine neutrophils incubated for 2 hrs at 37° C. either in the absence or presence of rubrene-DNase 1 nanoparticles at a concentration of ˜0.4 μg/mL of rubrene. FIG. 6B shows quantification of % positive cells for rubrene-DNase 1 nanoparticles over 1.5 hrs. FIG. 6C shows viability of neutrophils over 1.5 hr incubation at either 4° C. or 37° C. Geometric mean fluorescence intensity of neutrophils incubated with rubrene DNase 1 nanoparticles. Data represents N=3-4 biological replicates with N=1-2 technical replicates. Data is presented as the mean±S.E.M. % Cells positive were analyzed via multiple t-test comparison of row-by-row. ***=P<0.001, ****=P<0.0001. FIG. 6D shows the effect of internalization of DNase1-nanoparticles on neutrophil stimulation. Murine bone marrow derived neutrophils were incubated with DNase-1-nanoparticles for 2 hr (˜1 U/mL), and subsequently were stimulated with PMA (100 nM; middle bars), or PMA (100 nM) and ionomycin (1 μM; right bars). The oxidative burst was measured by the Abcam DHE ROS kit (Cat #Ab236206). Data represents 2 biological replicates conducted with 2 technical replicates. Data is presented as mean±S.E.M, and was analyzed by factorial ANOVA.

FIGS. 7A-7C show DNase1-NP loaded neutrophils migrate inside an in vitro murine blood clot. FIG. 7A is a schematic overview of experimental protocol. Briefly, murine bone marrow derived neutrophils were incubated with rubrene containing DNase1-NPs (˜0.4 μg/mL of rubrene) for 2 hrs at 37° C. Following this, murine venous blood was isolated and an in vitro blood clot was formed via recalcification and treatment with Tissue Factor and Platelet Activating Factor. 20 minutes or 60 minutes following clot formation, exogenous DNAse1-NP loaded neutrophils were added to the blood clot and incubated overnight. The clot was then retrieved and prepared as a fixed, individual cell suspension for analysis by flow cytometry. FIG. 7B shows 98% of isolated Ly6G-AF647 labelled murine bone marrow derived neutrophils were positive for rubrene-containing DNase-1 NPs following 2 hr incubation. Isolated neutrophils were utilized to gate the neutrophil population in SSC-A vs FSC-A. FIG. 7C shows exogenous neutrophil populations were detected in blood clot single cell suspensions. The single cell suspension was gated to cut off cell debris, then eliminate doublets and aggregates, and then neutrophils as determined by the previously isolated population in FIG. 7B, and then finally the positive population of Ly6G stained neutrophils was selected.

FIGS. 8A-8C show formation of the BSA-coated DNase 1 nanoparticle. FIG. 8A shows normalized intensity distribution size measurement via dynamic light scattering of the BSA coated inverse nanocarrier. 15 mg/mL of BSA was mixed directly with the inverse nanocarrier in the CIJ. FIG. 8B depicts the stability of the BSA coated inverse nanocarrier at pH 5 at 4° C. over 120 hr. FIG. 8C depicts the stability of the BSA coated inverse nanocarrier at pH 5 at 37° C. over 120 hr.

FIG. 9 is a schematic illustrating the iFNP processing steps.

FIG. 10 is a schematic illustration of neutrophil-mediated delivery of nano-encapsulated thrombolytic therapies for intra-clot delivery.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments±1%, in some embodiments±0.5%, and in some embodiments±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

### II. Subjects

The subject treated, screened, tested, or from which a sample is taken, is desirably a human subject, although it is to be understood that the principles of the disclosed subject matter indicate that the compositions and methods are effective with respect to invertebrate and to all vertebrate species, including mammals, which are intended to be included in the term “subject”. Moreover, a mammal is understood to include any mammalian species in which screening is desirable, particularly agricultural and domestic mammalian species.

The disclosed compositions, formulations, therapeutics and methods of using the same are particularly useful in the treatment of warm-blooded vertebrates. Thus, the presently disclosed subject matter concerns mammals and birds.

More particularly, provided herein is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, provided herein is the treatment of livestock, including, but not limited to, domesticated swine (pigs and hogs), ruminants, horses, poultry, and the like.

In some embodiments, the subject to be used in accordance with the presently disclosed subject matter is a subject in need of treatment and/or diagnosis. In some embodiments, a subject can have or be believed to be suffering from thrombosis or other related condition or disease, or any inflammation-associated disease, condition or phenotype.

### III. Therapeutic Formulations and Method of Treatment

Disclosed herein are therapeutic compositions and methods for treating a subject, the therapeutic compositions comprising a nanoparticle (in some embodiments a polymeric nanoparticle) encapsulating an active agent. In some aspects they further comprise a functionalized delivery vehicle for targeted delivery of the polymeric nanoparticle to a target tissue in the subject. In some embodiments, the active agent is releasable from the delivery vehicle at the target tissue. In some embodiments, and as disclosed herein in more detail, the active agent comprises a small molecule, nucleic acid, therapeutic protein and/or an active enzyme, optionally wherein the active agent comprises Deoxyribonuclease 1 (DNase 1), optionally wherein the active agent comprises an mRNA, e.g. an mRNA encoding for tPA or DNase 1.

More particularly, in some embodiments, disclosed herein are methods, compositions, formulations and approaches to therapeutically target and degrade neutrophil extracellular traps (NETs). NETs are an extracellular network of decondensed chromatin which are present in large quantities in human venous thrombus samples^{6, 7}, i.e. blood clots. Recent experimental and clinical data have increasingly shown that NETs contribute to structural changes in thrombi⁷⁻¹⁰. Additionally, NETs are reported to be pro-thrombotic as well as anti-fibrinolytic^{8, 11-14}, working synergistically with fibrin to render thrombi increasingly resistant to lysis with time¹⁴⁻¹⁶. Importantly, NETs have not been shown to be involved in critical hemostatic mechanisms, thus, in contrast to tPA, targeting the destruction of NETs is not predicted to result in bleeding side-effects⁷. To therapeutically target NETs the present disclosure provides, at least in some embodiments, for the delivery of an enzyme, e.g. Deoxyribonuclease 1 (DNase 1); an endonuclease that cleaves DNA in the extracellular space.

Nano- and therapeutic delivery technologies have the potential to offer strategies to overcome issues of poor therapeutic accumulation in thrombi. However, prior to the instant disclosure no therapeutic approach or method has been successfully developed to target NETs using such delivery technologies. Provided herein is the encapsulation of DNase 1 in polymeric nanoparticles, utilizing a next generation nanoparticle synthesis technology: inverse Flash Nanopreciptation (iFNP). Encapsulation of therapies into nanoparticles can confer unique, engineered properties that enable them to behave differently in vivo. Although several techniques currently exist for synthesizing nanoparticles, many techniques are not able to be industrially scaled up, exhibit poor drug-loading efficiency (leading to difficulty achieving a therapeutically relevant dose for humans), and poor homogeneity of nanoparticle production. iFNP uniquely addresses these challenges. In this process, nanoparticles are generated that contain biologic therapies in the core of the particle, surrounded by a protective layer of polymers that can also be functionalized to exploit ligand-receptor interactions. In some embodiments, the technology involves the rapid mixing of high-velocity streams of liquid, containing the polymers and therapies, in a specially engineered device (e.g. a micro inlet vortex mixer (MIVM) or confined impinging jet mixer (CIJ)). The rapidity of the mixing results in robust generation of homogenous nanoparticles (i.e. nanoparticles of substantially similar size and therapeutic loading). The diameter of the nanoparticle and the amount of therapeutic loaded into the nanoparticles can be tuned by adjusting the ratios of polymer to therapeutic in the liquid streams. To scale up, the channel diameters, stream flowrates and overall mixer device size can be increased appropriately to maintain sufficient micromixing (i.e. sufficient Reynolds numbers). FNP is well established for the encapsulation of pharmacologics that are poorly water-soluble, such as the chemotherapeutic, paclitaxel¹⁷, and antioxidants such as Vitamin-E¹⁸. For the encapsulation of highly water-soluble drugs and biologics, one must use iFNP. Thus far, iFNP has been employed to encapsulate horse radish peroxidase, the antibiotic Vancomycin, RNA and peptides¹⁹. Notably, cargo loading of these biologics was 5-15× higher than typical values obtained for liposomes and polymersomes (9-27 wt % vs<2%)¹⁹. Surprisingly, iFNP has thus far not been reported to encapsulate any therapeutic biologic with a biological application that maintains its biological activity upon administration to a subject. However, for the first time the present disclosure provides an iFNP protocol to generate nanoparticles encapsulating DNase 1. These particles are characterized for their size, polydispersity, encapsulation efficiency, loading capacity and release of active DNase 1. As shown and disclosed herein, these nanoparticles are capable of releasing DNase 1 to the extracellular space where it can degrade NETs.

DNase 1, or Deoxyribonuclease I, or DNase I, is an endonuclease of the DNase family coded by the human gene DNASE1. DNase 1 is a nuclease that cleaves DNA preferentially at phosphodiester linkages adjacent to a pyrimidine nucleotide, yielding 5′-phosphate-terminated polynucleotides with a free hydroxyl group on position 3′, on average producing tetranucleotides. It acts on single-stranded DNA, double-stranded DNA, and chromatin. In addition to its role as a waste-management endonuclease, it has been suggested to be one of the deoxyribonucleases responsible for DNA fragmentation during apoptosis. The structure and sequences of DNase 1 are further defined by Entrez 1773, UniProt P24855, NCBI Reference Sequence: NM_005223.4, and NCBI Reference Sequence: NP_005214.2.d.

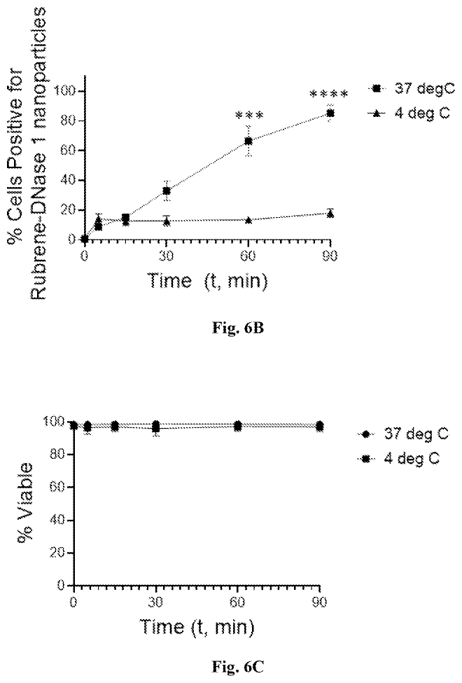

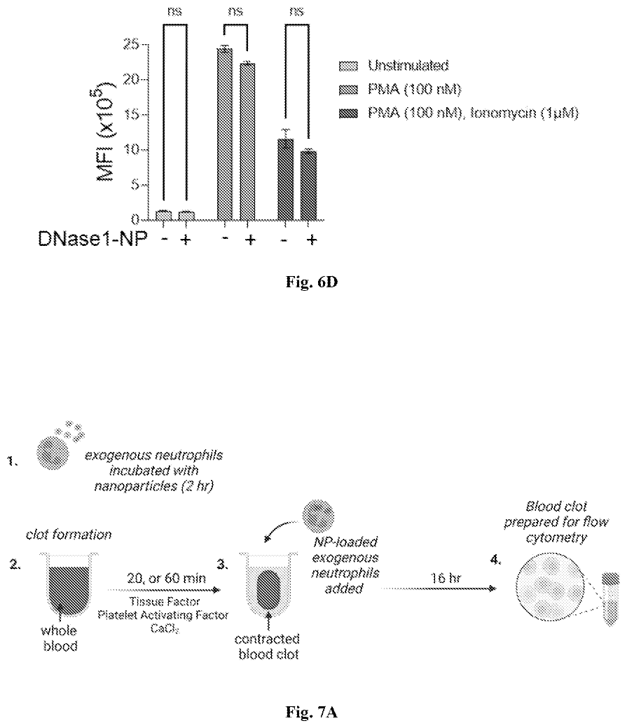

To direct these DNase1-nanoparticles to sites of venous thrombi, experiments were conducted to test the use of neutrophils as cell carriers. Neutrophils are among the first cells recruited to a growing thrombus and are then central to its progression. They are actively recruited and migrate into the milieu of thrombi via platelet-receptor interactions as well as signaling molecules. Moreover, the density of neutrophils inside a thrombus increases steadily with thrombus age. Employing neutrophils as a delivery vehicle was envisioned to address the issue of clot penetration and could circumvent the problem of bleeding associated with systemic tPA administration. Presented herein is data showing the surprisingly rapid internalization of the disclosed DNase1-nanoparticles by murine bone marrow derived neutrophils, without significant effects on their viability. See the illustration in FIG. 6A-C10.

The studies summarized herein support that nanoparticle encapsulation of biologics and active agents such as DNase 1, coupled with cell-mediated delivery represent a surprisingly effective and innovative tool to address poor therapeutic accumulation, which may be applied to venous thrombosis, among other indications and conditions. Biologic encapsulation techniques include, but are not limited to, the next-generation nanoparticle synthesis technique, iFNP.

While iFNP is an advanced method for making biologic-loaded nanoparticles, and in some embodiments a preferred approach, other nanoparticle synthesis processes can be used in accordance with the present disclosure.

By way of example, and not limitation, for making polymeric nanoparticles loaded with biologics, a coacervation process can be used.³¹ As an example of a coacervation process, first, proteins are mixed with a polymer solution, followed by a change in the solution property (e.g. pH, ionic strength), which induces a phase separation, forming nanoparticles.³² For making polymeric nanoparticles loaded with biologics, an emulsification process (e.g. homogenization) can be used.^{33,34} This technique involves dissolving a polymer and protein in a water-immiscible solvent. The solution is then emulsified in an aqueous phase to form droplets. As the solvent evaporates, nanoparticles containing the protein are left behind.³⁵ For making polymeric nanoparticles loaded with biologics, a layer-by-layer assembly process can be used.^{36,37,38} This method involves the sequential deposition of layers of oppositely charged polyelectrolytes onto a protein core. This layering process creates a shell around the protein, forming protein-loaded nanoparticles. For making polymeric nanoparticles loaded with biologics, a spray drying process can be used.^{39,40} For example, A protein-polymer mixture is atomized into fine droplets, which are then dried to form solid protein-loaded nanoparticles.⁴¹ For making polymeric nanoparticles loaded with biologics, a spray freeze-drying process can be used.^{42,43} For example, a protein-polymer mixture is atomized into fine droplets, which are then frozen and lyophilized to form solid protein-loaded nanoparticles.⁴⁴ This list of processes for polymeric nanoparticle synthesis is illustrative and not intended to cover all possible methods.

Still yet, by way of example and not limitation, for making lipid based nanoparticles loaded with biologics, many synthesis processes exist.⁴⁵ This is not an exhaustive list. For making lipid nanoparticle loaded with biologics, a lipid film hydration technique can be used.⁴⁶ As an example process, lipids are dissolved in an organic solvent to create a thin lipid film. The biologic material is then added to the film, followed by hydration with an aqueous solution, resulting in the formation of lipid nanoparticles. The lipid nanoparticles can then be extruded to attain the desired size distribution. For making lipid nanoparticles loaded with biologics, an emulsification process (e.g. homogenization) can be used.⁴⁷ Similar to the method for polymer nanoparticles, lipids and biologics are dissolved in an organic solvent and emulsified in an aqueous phase. The organic solvent is then evaporated to form lipid nanoparticles encapsulating the biologic cargo. For making lipid nanoparticle loaded with biologics, a self-assembly process induced by solvent quality shifting can be used. In this method, a solution containing lipids and biologics in an organic solvent is rapidly mixed with an aqueous phase. The mixing can be done using a microfluidic device,⁴⁸ a T-mixer, an impinging jet mixer,⁴⁹ a multi-inlet vortex mixer,⁵⁰ a rotor-stator, or by injection.⁵¹ This induces the self-assembly of lipids into nanoparticles that encapsulate the biologic payload.

Thus, in some embodiments provided herein are therapeutic compositions for treating a subject, the therapeutic compositions comprising a polymeric nanoparticle encapsulating an active agent. In some aspects they further comprise a functionalized delivery vehicle for targeted delivery of the polymeric nanoparticle to a target tissue in the subject. In some embodiments, the active agent is releasable from the delivery vehicle at the target tissue. In some embodiments, and as disclosed herein in more detail, the active agent comprises a small molecule, nucleic acid, therapeutic protein and/or an active enzyme, optionally wherein the active agent comprises Deoxyribonuclease 1 (DNase 1), optionally wherein the active agent comprises an mRNA, e.g. an mRNA encoding for tPA or DNase 1.

In some aspects, the functionalized delivery vehicle for targeted delivery of the polymeric nanoparticle to a target tissue in the subject comprises a cell or cellular component for cell-mediated delivery of the nanoparticle. Optionally, the cell or cellular component for cell-mediated delivery of the nanoparticle comprises an immune cell, optionally a macrophage or neutrophil, optionally any autologous cell. In some aspects, the functionalized delivery vehicle for targeted delivery of the polymeric nanoparticle to a target tissue in the subject comprises a delivery vehicle decorated with ligands with an affinity to infiltrating neutrophils.

Notably, the active agent maintains activity upon release from the delivery vehicle at the target tissue, optionally wherein the active agent comprises an enzyme with maintained enzymatic activity upon release at the target tissue.

In some embodiments, the nanoparticle is surrounded by a protective layer of polymers, optionally one, two or three layers of polymers. Alternatively, and a surprising finding as disclosed herein, in some embodiments at least one of the layers of polymers is substituted for a layer of protein, optionally wherein the layer of protein comprises albumin, optionally wherein the layer of protein stabilizes the nanoparticle. The protective layer of polymers can comprise one or more layers of polyethylene glycol (PEG) or similar aqueous stabilizing polymer (ex. poly(2-ethyl-2-oxazoline), poly(carboxybetaine), poly(sulfobetaine), poly(vinyl alcohol), poly(vinylpyrrolidone), and hyaluronic acid).

In some aspects, the nanoparticle comprises a poly-acrylic acid, complexed with spermine and/or triethylamine, and coated one or more layers of PLA and PEG. In some aspects, the nanoparticle comprises of a poly(acid) (e.g. poly(aspartic acid), poly(glutamic acid), poly(itaconic acid), poly(malic acid)) complexed with a small molecule polyamines (e.g. spermine, putrescine, cadaverine) or polymer-based poly(amine) (e.g. poly(ethylenimine, poly(propylamine), poly(lysine), poly(arginine) poly(histidine)), coated with one or more layers of a hydrophobic polymer layer (ex. PLA, poly(glycolic acid), poly(lactic-co-glycolic acid), poly(caprolactone), poly(propylene fumarate), poly(ortho-esters), poly(caprolactam), poly(anhydrides)) and a protective layer (ex, PEG, poly(2-ethyl-2-oxazoline), poly(carboxybetaine), poly(sulfobetaine), poly(vinyl alcohol), poly(vinylpyrrolidone), and hyaluronic acid), albumin). Additionally, in some applications the nanoparticle comprises dual encapsulated active agents, optionally a biologic and a small molecule.

In at least one preferred embodiment, provided is a therapeutic composition for targeting the destruction of a neutrophil extracellular trap (NET) in a venous thrombus in a subject, the therapeutic composition comprising a polymeric nanoparticle encapsulating a Deoxyribonuclease 1 (DNase 1) enzyme, and a cell carrier for targeted delivery of the polymeric nanoparticle to a venous thrombus in the subject, the cell carrier comprising a neutrophil, wherein the DNase 1 is releasable from the polymeric nanoparticle at the venous thrombus, wherein the DNase 1 maintains activity upon release. In such a composition the polymeric nanoparticle can comprise a layer of polyethylene glycol (PEG) and/or a layer of albumin. The therapeutic composition can further comprise any suitable stabilizer, additive or excipient to ensure the stability and/or maintenance of activity of the biologic or active agent. By way of example and not limitation, CaCl₂) or other stabilizing additives can be added to ensure in the maintenance of DNAse 1 activity upon release.

Correspondingly, provided in some embodiments are methods of treating a condition in a subject in need of treatment, the methods comprising administering to a subject a therapeutic composition as disclosed herein, whereby the subject's condition is treated. By way of example and not limitation, the subject can be suffering from an inflammatory condition, wherein the inflammatory condition recruits neutrophils and/or macrophages, whereby administration of the therapeutic composition comprising a functionalized delivery vehicle or cell carrier targets the therapeutic composition to the site of the inflammatory condition. In some embodiments, the subject is suffering from a venous thrombus, deep vein thrombosis, or a related blood clotting disease and/or condition. Administration of the therapeutic composition targets destruction of a neutrophil extracellular trap (NET) in a venous thrombus of the subject, optionally wherein intra-thrombus delivery of a NET-degrading enzyme, e.g. DNase 1, confers improved thrombolysis of venous thrombi in the subject without impacting surrounding tissues. In such methods, the therapeutic composition confers intra-thrombus delivery of a thrombolytic or anti-thrombotic enzyme to a hardened thrombus with greater penetration of the thrombus as compared to other treatments, whereby the therapeutic composition confers greater thrombolysis of a thrombus with fewer side-effects.

Finally, as explained further in the working Examples, also provided herein are methods of making a polymeric nanoparticle for encapsulation of one or more active agents, the method comprising inverse Flash Nanoprecipitation (iFNP) using an aqueous CaCl₂) solution, dioxane and triethylamine to maintain enzyme activity, with stabilization of a nanoparticle with polymer or a protein instead of a polymer.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-6

### General Materials

Poly(D,L-lactide-block-acrylic acid) (9k-9k, sigma Aldrich, 802190-1G), Methoxy poly(ethylene glycol)-b-poly(D,L-lactide) (5k-5k, Sigma Aldrich, 900658-500MG), Poly acrylic acid homopolymer (Sigma Aldrich, 9003-01-04), Poly(L-lactide)-PEG-Maleimide (PLLA-PEG-Mal, 5k-5k, Nanosoft Polymers), Rubrene (Sigma Aldrich, 554073-100MG), Triethylamine (Thermo Fisher, 04884100), DNase 1 (Sigma Aldrich, 10104159001), spermine (Sigma Aldrich, AAL11956203). Chloroform (CHCl₃, Thermo Fisher, AC423550025), 1,4-dioxane (Fisher Scientific, AC408820025), DMSO (Fisher Scientific, D128-1), Methanol (HPLC Grade, VWR, EM-MX0475-1), THF (Fisher Scientific, T425-4), Glacial Acetic Acid (Fisher Scientific, A38S-500), Molecular biology grade nuclease free water (VWR, 97062-794), RPMI 1640 media (11875135, Gibco), Heat-inactivated fetal bovine serum (FBS) (16140071; Gibco), Penicillin-Streptomycin 10,000 U/mL (15140122, Gibco), Phorbol 12-myristate 13-acetate (PMA, Sigma Aldrich P8139-5MG), Paraformaldehyde (158127; Sigma-Aldrich), 10×PBS (20-134; Apex Bioresearch Products, San Diego, CA), Calcium Chloride dihydrate (Fisher Scientific, C79-500), Magnesium Chloride Hexahydrate (Fisher Scientific, M33-500), Tris-HCl (Sigma Aldrich, T3253-1KG), Citric Acid (Sigma Aldrich, 251275-500G), Bovine Serum Albumin (Sigma Aldrich, A9418-5G), HBSS, no calcium, no magnesium, no phenol red (Thermo Fisher, Gibco, 14175095), Deoxyribonucleic acid sodium salt from salmon testes (Sigma-Aldrich, D1626-5G), 40% Acrylamide (Sigma Aldrich, A2792), TEMED (Sigma Aldrich, 50-197-8483), Ammonium persulfate (Sigma Aldrich, A3678).

### DNase 1 Activity Assay

DNase 1 activity in solution was measured with the commercially available fluorometric plate-reader assay from Abcam (ab23406). Briefly, solutions containing 5 mg/mL of DNase 1 were diluted to 0.1-0.5 μg/mL in molecular biology grade water to be used as samples. In the case where organic solvents were used for the original suspension, a control was run containing a similar percentage of final organic solvent with control DNase 1 to ensure that this did not adversely affect the plate reader assay. Preparation of the samples and a standard curve were performed as per the protocol instructions in the product insert. Fluorescence was measured (Ex/Em=651/681 nm) in kinetic mode, every 30 seconds, for 90 minutes at 37° C. DNase 1 activity from DNase 1 dissolved in organic solvent mixtures, or poly acrylic acid/spermine/triethylamine mixtures, was recorded as a percentage of the activity for DNase 1 from the same lot, dissolved in water.

Measurements with Dynamic Light Scattering

Measurements for nanoparticle size with intensity distribution and correlation function were performed with Dynamic Light Scattering (DLS) and were carried out with a Zetasizer NanoZS (Malvern Panalytical, Ltd, UK). Size measurements were conducted at a 173-degree scattering angle, with the appropriate settings for the solvent. Measurements were conducted at least 3 times with 30 s to 240 s of equilibration time. For measurements in organic solvents, we employed the Malvern Panalytical Sq Cell W Cap 12 mm O.D. glass cuvette (Fisher scientific, NC0631452), whilst measurements in aqueous solvents were recorded in MalvemPanalyticalInc 40 μL Cuvettes (Fisher Scientific, NC0628994). Samples were typically diluted 10-fold for measurements into appropriate solvents (Chloroform, 1,4-dioxane or 0.02 μm filtered water or 1×PBS).

### Generation of DNase 1 Nanoparticles

DNase1 nanoparticles (DNase 1-NPs) were synthesized via inverse flash nanoprecipitation (iFNP) with methods adapted from previous literature^{19, 20}. FIG. 9 provides a schematic illustration of at least one exemplary embodiment of iFNP processing steps.

Step 1. Inverse nanocarrier: Briefly, a micro multi inlet vortex mixer (MIVM) was fitted with a total of 4 1 mL syringes: 2 syringes containing 500 μL of Chloroform (CHCl₃, Thermo Fisher, AC423550025), 1 syringe containing 500 μL of 20 mg/mL of Poly(D,L-lactide-block-acrylic acid) (9k-9k, Sigma Aldrich, 802190-1G) dissolved in 90% 1,4-dioxane (Fisher Scientific, AC408820025) and 10% 0.1M Tris-HCl pH 7.8 buffer, and finally 1 syringe containing 50 μL of DNase 1 (10 mg/mL in 0.1 M Tris-HCl, pH 7.8, containing 10 mM CaCl₂)), and 450 μL of 1,4-Dioxane. The syringes were depressed rapidly into a quench bath (8 mL volume containing 2 mg/mL spermine, 635 μL Triethylamine and CHCl₃) and stirred rapidly for 5 minutes. The nanoparticle solution in CHCl₃ was then extracted into 4 mL of 150 mM NaCl, pH 6 for 30 minutes at a slow rocking speed. The aqueous layer was removed, and the organic layer was centrifuged at 1000×g for 10 minutes at 4° C. Emulsions of the nanoparticle with aqueous layer that were difficult to remove were left aside. The organic layer was transferred to a 50 mL glass round bottom flask and 20 mg of Methoxy poly(ethylene glycol)-b-poly(D,L-lactide) (5k-5k, Sigma Aldrich, 900658-500 MG) was added. The solution was then solvent-swapped into 1,4-dioxane by adding 10 mL of 1,4-dioxane and removing the solvent by rotary-evaporation till 1 mL remained. This was repeated three times.

Step 2. Aqueous stabilized nanocarrier: 1 mL of the inverse nanocarrier in 1,4-dioxane and containing 20 mg of methoxy-PEG-PLA polymer was drawn into a 1 mL syringe and fitted to a confined impinging jet mixer (CIJ). 1 mL of water was fitted to the other inlet. The syringes were depressed rapidly into 8 mL of water and stirred rapidly for 5 minutes. The nanoparticle suspension was then dialyzed overnight against DI water (Biotech CE Dialysis Tubing 300 KD 16 mm, VWR, 89068-798). To generate fluorescent nanoparticles, 2 mg of rubrene (sigma Aldrich 554073-100 MG) was added to the organic solvent just prior to injecting the solvent streams through the CIJ.

Generation of DNase 1 Nanoparticles Coated with BSA

Bio-conjugation with Maleimide-thiol chemistry: The above steps were performed exactly as described above, however in Step 2, 10 mol % of the Methoxy-PEG-PDLLA was replaced with Poly(L-lactide)-PEG-Maleimide (PLLA-PEG-Mal). The aqueous stabilized nanoparticles were quenched into 8 ml of a 0.5 mM citric acid buffer at pH 5. These nanoparticles were incubated, with light rotation, overnight at room temperature, with BSA in 0.15M NaCl to achieve a molar thiol ratio of 4 to 1 Peg-Maleimide. Nanoparticles were diluted 2-fold into the BSA solution. Thiols on the BSA were initially measured with the Ellmans' reagent plate reader assay to determine thiol content in BSA solutions. Free BSA was then removed by dialysis against DI water with a total of 6 changes of dialysate (Biotech CE Dialysis Tubing 300 KD 16 mm, VWR, 89068-798).

Direct adsorption with FNP: 1 mL of the inverse nanocarrier in 1,4-dioxane was drawn into a 1 mL syringe and fitted to a confined impinging jet mixer (CIJ). 1 mL of water, containing an excess of either native or denatured BSA (1.5-2-fold relative to total dry mass of the inverse nanocarrier) was fitted to the other inlet. The syringes were depressed rapidly into 8 mL of water and stirred rapidly for 5 minutes. The nanoparticle suspension was then dialyzed overnight against DI water (Biotech CE Dialysis Tubing 300 KD 16 mm, VWR, 89068-798). To generate fluorescent nanoparticles, 2 mg of rubrene (sigma Aldrich 554073-100 MG) was added to the organic solvent just prior to injecting the solvent streams through the CIJ. To denature BSA it was heated to 85° C. for 20 minutes and then cooled on ice.

### Encapsulation Efficiency

Encapsulation Efficiency was broadly determined by measuring the amount of DNase 1 protein recovered into the aqueous extract phase following formation of the inverse nanocarrier. Inverse nanocarriers containing DNase 1 were synthesized as described, but in the absence of spermine and triethylamine, and then extracted with 0.15M NaCl solution. Controls included: inverse nanocarriers without DNase 1 added, DNase 1 alone with no stabilizing polymer added, aqueous extract buffer alone. To deduct the Encapsulation Efficiency, we utilized the following equation:

$$Encapsulation Efficiency = 100 - ( 100 \times \frac{DNase {1}_{inverse}}{DNase {1}_{max}} )$$

DNase 1inverse=the amount of DNase 1 recovered into the aqueous extract phase following formation of DNase 1 containing inverse nanocarriers

DNase 1max=the amount of DNase 1 recovered into the aqueous extract phase following DNase 1 passing through the MIVM with no stabilizing BCP added (no inverse nanocarrier formed).

### Micro BCA Assay

The aqueous extraction phase was measured for protein using the commercially available micro BCA assay (Thermo Fisher, 23235). Instructions as per the user's manual were followed and sample absorbances were measured at 562 nm. Measurements were made in triplicate with three independent batches of nanoparticles synthesized.

### Protein Absorbance at 280 nm

A second, orthogonal, measure was utilized to confirm Encapsulation Efficiency. We measured protein absorbance at 280 nm using the Duetta Fluorescence and Absorbance Spectrometer. Measurements were made in triplicate with three independent batches of nanoparticles synthesized.

### Loading Capacity and Loss to Extraction of the Inverse Nanocarrier

To determine the Loading Capacity and loss to extraction of the inverse nanocarrier we used a Thermogravimetric Analyzer (TGA, TA instruments). Inverse nanocarriers containing DNase 1 were synthesized as described, but in the absence of spermine and triethylamine, and then extracted with 0.15M NaCl solution. 90 μL of the inverse nanocarrier solution in chloroform was added to the TGA pan prior to and following the extraction step, and the dry mass recorded. Loading capacity was determined by employing the following equation:

$$Loading Capacity = ( \frac{DNase {1}_{max} \times EE}{Nanoparticle Mass} ) \times 10$$

DNase 1max=the amount of DNase 1 recovered into the aqueous extract phase following DNase 1 passing through the MIVM with no stabilizing BCP added (no inverse nanocarrier formed).

### EE=Encapsulation Efficiency

Nanoparticle Mass=total dry mass of the nanoparticle as calculated from measurements with the TGA. Mass in 90 μL was extrapolated to calculate the total mass based on the total volume of the nanoparticle suspension.

DNase 1-nanoparticle loading in aqueous stabilized PEG-coated nanoparticle Loading of the final aqueous stabilized PEG-coated nanoparticle was confirmed by gel electrophoresis followed by Silver Stain. Briefly, following dialysis, nanoparticles were diluted with 6×SDS loading buffer (Fisher Scientific, 50-196-785) and boiled (10 min, 90° C.). A series of DNase 1 standards were similarly treated. These were loaded into a Sodium dodecyl sulfate (SDS)—polyacrylamide gel (4% (v/v) stacking gels and 10% (v/v) running gels). Electrophoresis was carried out at 120 V using Tris/glycine electrophoresis buffer (25 mM Tris, 192 mM glycine, 0.1% (w/v) SDS, pH 8.7), just until the protein band had surpassed the stacking gel. To stain for the protein content, we used the commercially available Silver Stain Plus Kit, and followed the protocol instructions supplied (Biorad, 1610449). Gels were imaged using an Azure Biosystems Gel Doc imaging system.

### Detection of DNase 1 Activity Via Denaturing Polyacrylamide Gel Electrophoresis Zymography

We performed this assay as previously described in Jimenez-Alcázar and co-authors²¹, and Rosenthal and Lacks²², with adaptations. Briefly, nanoparticles were degraded by diluting them 2-fold into a solution with a final concentration of 5% SDS and 0.1M NaOH, and then stirred rapidly at room temperature for 48 hrs. This sample was then diluted in water to represent ˜100 pg of DNase 1. DNase 1 standards were prepared in water representing 51, 102 and 204 pg of DNase 1. The sample and standards were diluted with 6×SDS sample loading buffer, and then boiled for 10 minutes before loading onto the gels.

Sodium dodecyl sulfate (SDS)—polyacrylamide gels were prepared with 4% (v/v) stacking gels without DNA and 10% (v/v) running gels containing 100 μg/ml of denatured salmon testes DNA. DNA was first dissolved in water (30-60′ sonication) then boiled (10 min, 90° C.), and placed on ice before addition to gel reagents. Electrophoresis was carried out at 120 V using Tris/glycine electrophoresis buffer (25 mM Tris, 192 mM glycine, 0.1% (w/v) SDS, pH 8.7). After electrophoresis, SDS was removed by washing the gel twice with 10 mM Tris/HCl pH 7.8 for 30 min at 50° C. Proteins were refolded by incubating the gels overnight at 37° C. in a re-folding buffer containing 5% (w/v) milk powder, 10 mM Tris/HCl pH 7.8, 3 mM CaCl2), 3 mM MgCl2, 100 U/ml penicillin, and 100 μg/ml streptomycin. The gels were transferred to a refolding buffer without milk powder (10 mM Tris/HCl pH 7.8, 3 mM CaCl2), 3 mM MgCl2, 100 U/ml penicillin, and 100 μg/ml streptomycin) and incubated for additional 24 hrs at 37° C. The gels were then stained with for DNA with SYBR™ Safe DNA Gel Stain in 1×TAE (Life Technologies, S33111). Fluorescent images of the gels were recorded using a molecular gel imager.

DNase 1 Activity Assay with Single Radial Enzyme Diffusion (SRED)

DNase 1 activity was measured as previously described²¹ with minor alterations. 25 μg/mL of denatured DNA from salmon testes was dissolved in a buffer (10 mM Tris-HCl pH 7.8, 3 mM CaCl₂), 3 mM MgCl₂) with 1% w/v agarose. This was then heated and poured over glass slides and stored at room temperature until solidification. Wells of 1.0 mm in diameter were created using 1.0 mm diameter plastic tubing. 2 μL of DNase 1 solution (standards or sample diluted into water) were loaded into the wells. Gels were incubated for 4 hrs at 37° C. in a humid chamber. DNA was visualized by staining with SYBR™ Safe DNA Gel Stain in 1× TAE (Life Technologies, S33111). Gels were lifted off the glass slides, and DNA was visualized by fluorescence imaging of the gel.

### DNase 1 Release

450 μL of 10×buffer (0.1M Tris-HCl pH 8, 30 mM MgCl₂, 30 mM CaCl₂)) and 50 μL of DNAse 1 nanoparticles were added to a Dialysis tube-O-dialyzer, with a final volume of 500 μL. The dube-O-dialyzer was placed into 50 mL falcon tube containing 35 mL of release buffer (10 mM Tris-HCl pH 8, 3 mM MgCl2, 3 mM CaCl2)). The tubes were incubated on a plate shaker at 37° C. At 1 hr, 3 hr, 6 hr, 22 hr, 30 hr, 48 hr the tube-O-lyzer was moved to a new 50 mL falcon with 35 mL of fresh release buffer. Protein in th 35 mL of release buffer was concentrated using Pall centrifugal filters (20 mL, MWCO 1K and 10K mix). The final volume was recorded (around 3 mL per time point). Protein content was determined by measuring absorbance at 280 nm as described before using a calibration curve of pure DNAse 1 in 1×release buffer.

### Isolation of Bone Marrow Derived Neutrophils

Bone marrow derived neutrophils were isolated as previously described with minor alterations. Briefly, the femur and tibia of 10-14-week-old C57bl/6 mice were excised, and the bone marrow collected into cold HBSS prior to red blood cell lysis with hypotonic sodium chloride (0.2%), followed by addition of NaCl solution (1.6%). Cells were strained (40 μm nylon cell strainer) and then pelleted (300 g, 5 min) and resuspending in HBSS. Cells were counted and then neutrophils were isolated by magnetic separation with a mouse neutrophil isolation kit (Miltenyi, 130-097-658). The instructions were followed as per the kit protocol. A Midi Macs separator (Miltenyi, 130-042-302) with LS columns (Miltenyi, 130-042-401) was employed. Neutrophils were finally resuspended at ˜1×10⁶ cells/mL in 1×RPMI (no serum or antibiotics added). The typical yield from 1 mouse was ˜30×10⁶ bone marrow cells and ˜5×10⁶ neutrophils, with 98% viability. Cells were checked for initial cell count and viability with the Muse Count and Viability kit (Sigma Aldrich, Luminex MCH100104). Neutrophils were always used fresh and within 2 hrs of isolation, they were kept at 4° C. until use.

### Formation of In Vitro Blood Clot

Murine blood was isolated from 10-16 week old C57Bl/6J mice from the inferior vena cava into syringes containing 4% sodium citrate. A 96-well half-area flat-bottom plate (Fisher Scientific, Catalog #: CLS3695-25EA) was silanized with Sigmacote (Sigma Aldrich, Cat #: SL2-100 ML). In one well of a silanized 96-well half-area plate, the following were added to different corners of the well: 1 μL of Tissue Factor (Innovin, Fisher Scientific, final dilution is 1/9000), 1 μL of Platelet Activating Factor (Sigma Aldrich, Catalog #: 511075-1 MG, final concentration 50 μM), 1 μL of CaCl2) (Final Concentration, 30 mM). Blood was then added to a final volume of 80 μL. The plate was placed inside an incubator (37° C., 5% CO¬2) and the clot allowed to form over a 2-hour period. Fluorophore-labelled exogenous murine bone marrow derived neutrophils (10×10⁶ cells/mL, 80 μL, RPMI) were either added at the beginning of clot formation, or 20 min, or 60 min post clot initiation conditions. They were then incubated with the clot overnight at 37° C., 5% CO2.

The entire clot was prepared for flow cytometry by grinding the clot into 20 mL of HBSS and pelleting the cells (500×g, 5 min, RT) followed by simultaneous red blood cell lysis and fixation (Thermofisher, Catalog #00=5333-54).

### Flow Cytometry

Flow Cytometry was performed on the Thermo Fisher Attune N×T instrument and data acquired on the Attune N×T 2.6 software. Data was analyzed using FlowJo software. For staining we employed Anti mouse CD45-PeCy5 (Fisher Scientific, 15-0451-81, 1:3000, final ˜0.07 ug/mL) and the Violet live dead stain (Thermo Fisher, L34963, 1:1000). Cells were gated in the order of: cell granularity/size, singlet events, live cells, CD45+, rubrene+.

For blood clots, cells were gated in the order of: cell debris exclusion, singlet events, cell granularity/size for neutrophils, Ly6G positivity (AF-647). Exogenous neutrophils had been stained with Anti-ly6G AF647 (Biolegend, Clone 1A8, Cat #: 127609, 1/500 dilution).

### Nanoparticle Internalization by Murine Neutrophils

This method was adapted from previous literature²³. Briefly, 96 well U-bottom microplates (08-772-54, Fisher scientific) were treated overnight with 1% BSA/1×PBS. On the day of the experiment, 100,000 murine neutrophils in RPMI media (no serum) at 1×10⁶ cells/mL were added to the treated wells. 10 μL of rubrene-DNase 1-NPs (final concentration of ˜0.2 μg/mL rubrene) were added to the neutrophils and incubated either at 37° C. with 5% CO2 and humidity or 4° C. or up to 2 hours. Neutrophils were pelleted (300 g, 5 min), and washed with flow cytometry staining buffer (0.5% BSA in 1×PBS). Cells were pelleted and resuspended in 1×PBS and stained with the violet live/dead stain for 30 minutes, protected from light at room temperature. Cells were washed and then stained with CD45 antibody for 30 minutes at 4° C. Cells were washed and then fixed with 1% PFA in 1×PBS for 15 minutes at 4° C. Cells were stored overnight in 1×PBS at 4° C. and analyzed by flow cytometry within 48 hours.

### Stimulation of Oxidative Burst in Neutrophils

Oxidative burst of bone marrow derived neutrophils was measured using the Abcam DHE ROS kit (Cat #Ab236206). Neutrophils (1×106 cells/mL, RPMI) were incubated in the presence or absence of DNase1-nanoparticles (3.4 U/mL) for 2 hrs at 37° C., 5% CO¬2, and then pelleted and washed to remove unincorporated nanoparticles. These neutrophils were subsequently stimulated with PMA (100 nM) or PMA (100 nM) and ionomycin (1 μM) for 30 minutes at 37° C., 5% CO2. The instructions for the abeam kit were then followed to stain these cells for ROS production.

### Generation of Neutrophil Extracellular Traps

NET degradation was analyzed as previously described^{29,30}, with minor alterations. 50,000 purified murine neutrophils (250,000 cells/mL) in serum-free F12 media were seeded onto sterile 96 well glass bottom plates (VWR, 655891). To induce NET formation, neutrophils were treated with 100 nM PMA for 4 hrs at 37° C. with 5% CO2 and humidity. Wells were then washed three times with 1×PBS and stored in 1×PBS overnight at 4° C. DNase 1 or DNase 1-NPs at ˜1 U/mL of DNase 1 were added to wells in Tris-HCl, 10 mM pH 7.8, containing 3 mM CaCl2) and 3 mM MgCl2. Control wells were incubated with buffer alone. NET degradation was allowed to occur for up to 4 hrs. To stop NET degradation, wells were washed and treated with cold 2 mM EDTA for 15 min at 4° C. Well were washed, and then NETs fixed with 2% PFA in 1×PBS for 15 min in the fridge. Wells were washed and DNA stained with 2 μM Sytox Green (Thermo Fisher, S7020) for 15 min at room temperature. Wells were washed and stored until imaging in 1×PBS, protected from light at 4° C. Entire wells were imaged for GFP fluorescence with High Content Analysis Nikon Elements software using an inverted fluorescence microscope (Nikon Ti2 eclipse, Objective: Plan Apochromat Lambda 10×, NA=0.45, WD=4 mm). Analysis was performed in Image J. Firstly, a standardized well area was drawn that excluded the very edges of the well, and this region was duplicated. The same area was applied to all wells. The fluorescence was thresholded and the area of the thresholded fluorescence was measured. To restrict measurements to the filamentous NET structures, circular particles were counted, and the area of the particles measured. Area from spherical particles was deducted from the total initial area. The remaining area was considered to be that of filamentous NET structures.

TABLE 1
Dissolution of DNase 1 in organic solvent/aqueous
mixtures and its residual activity.

| | | | | % of |
|---|---|---|---|---|
| | Organic | CaCl₂ | Aqueous | Maximum |
| Solvent | (vol %) | (mM) | matrix | Activity |
| DMSO | 75 | 20 | H₂O | ~0 |
| Methanol | 50 | 10 | H₂O | * |
| | 90 | 10 | H₂O | * |
| Acetonitrile | 50 | 10 | H₂O | ~0 |
| Tetrahydrofuran | 50 | 10 | H₂O | ~0 |
| | 70 | 20 | H₂O | ~0 |
| | 80 | 20 | H₂O | ~0 |
| 1,4-Dioxane | 50 | 20 | 0.1M Tris | 98.8 ± 9.4 |
| | | | pH 8 | |

*Protein precipitated, pre-empting measurement of DNase 1 activity.
Footnotes:
DNase 1 was prepared to be a final concentration of 5 mg/mL in these organic solvent/aqueous mixture preparations. DNase 1 activity was measured using the fluorometric DNase 1 activity assay (ab23406), and activity represented as a percentage of the activity of DNase 1 dissolved at a similar concentration but in water.

### Example 1

### Inverse Nanocarrier Formation

The next-generation nanoparticle synthesis technology, iFNP^{19,20}, was used to encapsulate the protein DNase 1, with the goal of achieving (1) high encapsulation efficiency, (2) high loading capacity, and (3) employing a nano-formulation synthesis technique with proven scalability. An initial requirement of iFNP is the dissolution of the protein in a non-polar organic solvent/aqueous mixture. Several organic solvent/aqueous mixtures were tested for both solubility and maintenance of DNase 1 activity (Table 1) since such had not been done previously.

Surprisingly, DNase 1 activity was maintained even after formulation of the NPs, but only after significantly modifying the previously used methodology for iFNP. Calcium Chloride (CaCl2)) was included in the aqueous portion of all mixtures tested. These results indicated that, although DNase 1 was readily soluble in mixtures of DMSO/water, activity was completely lost. Alternatively, and unexpectedly, it was found that dissolution in a mixture of 1,4-dioxane and CaCl₂) aqueous buffer was feasible and could maintain activity at levels comparable to DNase 1 in an aqueous matrix. Indeed, in some embodiments it was surprising to find that to maintain DNase 1 enzymatic activity, the use of 1,4-dioxane and CaCl₂) containing buffer (or a similar buffer) in the iFNP process could be necessary. The use of 1,4-dioxane and CaCl₂) containing buffer in the process to preserve enzymatic activity is disclosed herein for the first time.

TABLE 2
Influence of CaCl₂ on DNase 1 activity
in Dioxane/aqueous mixture

| | | | % of |
|---|---|---|---|
| | Organic | CaCl₂ | Maximum |
| Solvent | (vol %) | (mM) | Activity |
| 1,4- | 90 | 0 | 25.3 |
| Dioxane | | 4 | 107.8 |
| | | 10 | 102.3 |
| | | 20 | 0 |
| | | 40 | 0 |

fluorometric DNase 1 activity assay (ab23406), and activity represented as a percentage of the activity of DNase 1 dissolved at a similar concentration but in water.

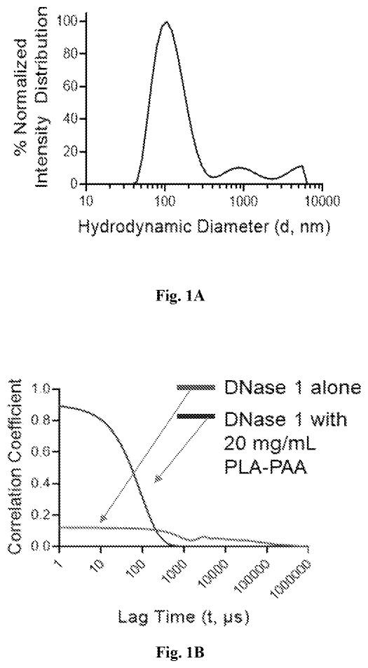

While utilizing CaCl₂) to enhance dissolution and help maintain activity, increasing the CaCl₂) content in the aqueous matrix was tested for its effect upon solubility and DNase 1 activity in a 1,4-dioxane mixture (Table 2). Interestingly, it was found that concentrations of 20 mM and higher resulted in a complete loss of DNase 1 activity. Moreover, concentrations above 40 mM resulted in phase separation of the aqueous phase from the organic phase. Thus, to form the inverse nanocarrier a solvent stream of 90% dioxane containing 0.1M Tris-HCl, pH 8, with 10 mM CaCl₂) was chosen for the DNase 1 protein. To form the inverse nanocarrier, hydrophilic cargo is initially encapsulated using an amphiphilic di-block co-polymer (BCP)^{20, 28}. In the present study, Poly(D,L-lactide-block-acrylic acid (PLA-PAA, 9k-9k, sigma Aldrich) was used. As the Poly-acrylic acid portion can be potentially cross-linked by the divalent cations present in CaCl2), the streams of protein and polymer were separated by use of a micro inlet vortex mixer (MIVM, Holland Applied Technologies). Hence, to initially encapsulate DNase 1 in an inverse nanocarrier, stabilized by a PLA corona, PLA-PAA (20 mg/mL) was dissolved in 90% 1,4-dioxane, containing 0.1M Tris-HCl buffer at pH 8. Meanwhile, DNase 1 was also dissolved in 90% dioxane at 1 mg/mL in 0.1M Tris-HCl buffer at pH 8, containing 10 mM CaCl₂). These two streams were turbulently micro-mixed in the MIVM against an antisolvent, chloroform. The resulting dispersion was quenched in a chloroform bath. A typical intensity-weighted size distribution for this formulation is shown in FIG. 1A. Additionally, the correlogram is shown (FIG. 1B) alongside a control without the BCP stabilizer, which had afforded visible precipitation of DNase 1, and poor particle formation. This highlights that the particles are not simply DNase 1 aggregates but are stabilized by BCP.

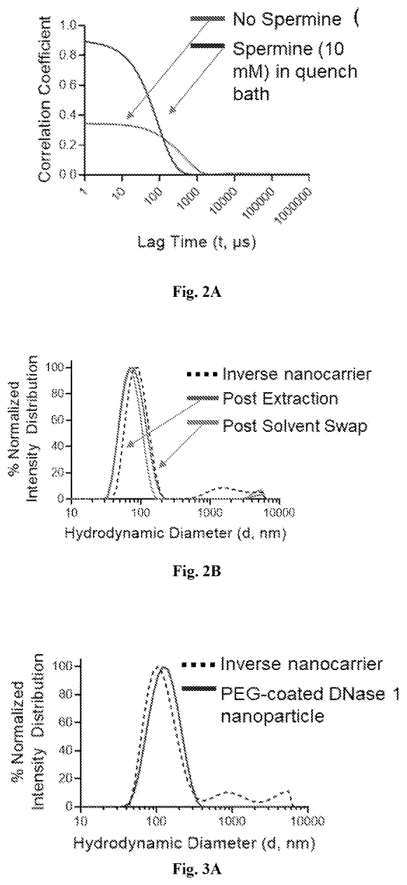

To stabilize the inverse nanocarrier for the further processing steps, ionic complexation was used with a polyamine, spermine. Spermine could be introduced either in the chloroform antisolvent stream or in the quench bath. It was found that inverse nanocarrier size and distribution was best preserved by including 10 mM spermine in the chloroform quench bath. To assess the strength of the cross-linking, a dynamic light scattering method was used, relying on comparing the correlation function of the inverse nanocarrier with and without spermine added²⁸. As shown in the correlation function in FIG. 2A, when the inverse nanocarriers were diluted 10-fold into 1,4-dioxane, those that were not stabilized by ionic complexation with spermine did not persist. It was possible to additionally dissolve these stabilized inverse nanocarriers in either DMSO or THF (data not shown) without significant swelling or adverse change to the correlation function. Thus, the inclusion of spermine allowed for maintaining nanoparticle size and polydispersity from the initial generation of the inverse nanocarrier, and throughout extraction and solvent swap with 1,4-dioxane, as shown in FIG. 2B. It was opted to continue to dissolve the nanocarriers in 1,4-dioxane to minimize potential contact of DNase 1 with adverse activity-killing solvents such as THF or DMSO. Using 1,4-dioxane for the coating step is disclosed herein for the first time.

As the core of the nanoparticle is, in some embodiments, poly-acrylic acid, complexed with spermine, efforts were undertaken to mimic the core conditions to approximate the pH of the core, and estimate the potential impact of this pH on DNase 1 activity (Table 3). The formulation was calculated to have a theoretical concentration of about 100 mg/mL of PAA in the core of the particle. By adding 10 mM spermine, the pH was raised to about 3.5, resulting in a loss of DNase 1 activity of about 60%. Raising the pH with increasing concentrations of triethylamine resulted in concomitant increases in DNase 1 activity. Based on these core condition approximation studies, a final concentration of 0.5 M Triethylamine (TEA) in the quench bath was chosen, with the goal of modulating the pH of the core to favor conditions that preserve DNase 1 Activity. The addition of TEA in the process to preserve enzymatic activity is disclosed herein for the first time.

TABLE 3
Modeling optimal conditions for DNase 1
activity in the inverse nanocarrier core

| PAA | Spermine | TEA | % Max | |
|---|---|---|---|---|
| mg/mL | (mM) | (M) | activity | pH |
| 50 | 0 | 0 | 18.90 | 3 |
| | 10 | 0 | 48.20 | 3.3 |
| 100 | 10 | 0 | 39.3 ± 3.9 | 3.5 |
| | | 0.2 | 38.5 ± 1.4 | 4 |
| | | 0.35 | 82.2 ± 2.4 | 4.5 |
| | | 0.5 | 102.2 ± 5 | 5 |
| 200 | 10 | 0.5 | 37.7 ± 0.6 | 4.5 |
| | | 0.75 | 89.5 ± 13.4 | 5 |

Footnotes:
PAA is Poly-acrylic acid, TEA is triethylamine. DNase 1 activity was measured using the fluorometric DNase 1 activity assay (ab23406), and activity represented as a percentage of the activity of DNase 1 dissolved at a similar concentration but in water. pH was measured using pH strips.

### Example 2

### Aqueous Stabilized Nanocarrier Formation

To form the aqueous stabilized nanoparticle, the inverse nanocarrier was coated with a PEGylated amphiphilic block co-polymer. To achieve this the well-established technique of Flash Nanoprecipitation (FNP)²⁰ was employed, which requires the dispersion of the nanocarrier in a water miscible organic solvent (such as 1,4-dioxane). As the formation of the initial inverse nanocarrier results in nanoparticles dispersed in chloroform along with excess spermine and triethylamine, an aqueous extraction was first performed with 150 mM sodium chloride to remove the residual 1,4-dioxane, spermine and triethylamine. Secondly, a solvent swap was performed with a rotary-evaporator to disperse the nanoparticles in 1,4-dioxane. Representative intensity distributions of the inverse nanocarrier prior to extraction, following extraction, and the solvent swap steps are shown in FIG. 2B.

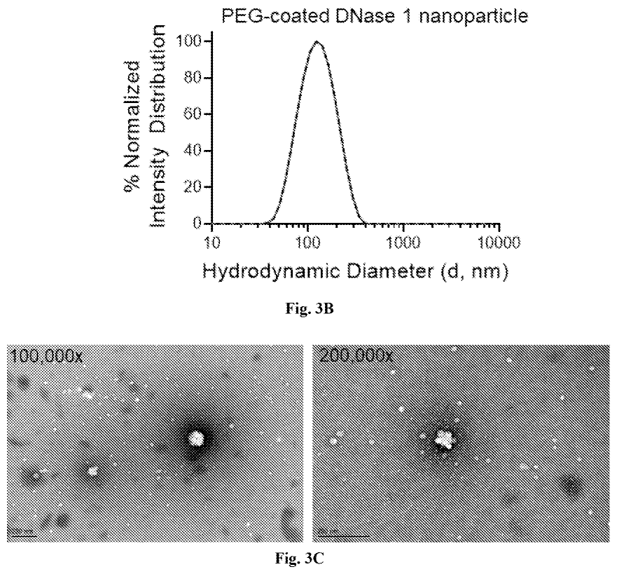

To coat the inverse nanocarrier with PEG, the inverse nanocarrier was dissolved in 1,4-dioxane alongside methoxy-PEG-PLA (20 mg). This polymer/nanocarrier mixture was rapidly mixed with an antisolvent, water, in a confined impinging jet mixer (CIJ) to generate PEG-coated DNase 1 nanoparticles. The intensity distribution of the inverse nanocarrier compared to the final PEG-coated DNase 1 nanoparticle can be seen in FIG. 3A. To make fluorescent particles, about 1 weight % of rubrene relative was added to the PEG BCP (0.2 mg), which did not significantly alter size or polydispersity of the PEG-coated DNase 1 nanoparticles, as shown in FIG. 3B. The results obtained by DLS were confirmed by TEM as shown in FIG. 3C (TEM of Rubrene-containing DNAse-1-nanoparticles), particles were adsorbed onto copper 400 mesh TEM grids were negatively stained with 2% uranyl acetate and imaged via TEM at 100,000× and 200,0000× magnification (scale bar=200 nm). A summary of the sizes and polydispersity indices of DNase 1 nanoparticles at each processing step of iFNP is shown in Table 4. While the fluorophore rubrene was used in this instance, other hydrophobic small molecules including active drug compounds could be encapsulated as would be appreciated by one of ordinary skill in the art. Weakly hydrophobic small molecules could also be encapsulated using the hydrophobic ion pairing approach.

TABLE 4
Summary of hydrodynamic diameter and polydispersity
of DNase 1 nanoparticles

| Stage of | | Size (hydrodynamic | Polydispersity |
|---|---|---|---|
| Processing | Solvent | diameter, nm) | Index (PDI) |
| Inverse nanocarrier | Chloroform | 106.1 ± 1.1 | 0.298 ± 0.017 |
| Post extraction | Chloroform | 89.5 ± 3.3 | 0.128 ± 0.014 |
| Post Solvent Swap | 1,4-dioxane | 72.0 ± 5.6 | 0.182 ± 0.012 |
| PEG-coated | Water | 128.1 ± 14.6 | 0.199 ± 0.069 |
| nanoparticle | | | |
| Post-dialysis | Water | 117.3 ± 9.0 | 0.200 |
| overnight in water | | | |
| PEG-coated | Water | 103.3 ± 6.7 | 0.201 ± 0.032 |
| nanoparticle | | | |
| containing rubrene | | | |

Data represents 2-5 independent batches of DNase-1 nanoparticles and 3 independent batches of rubrene-containing DNase-1 nanoparticles.

### Example 3

### Aqueous Stabilized Nanocarrier Formation with Aqueous Bovine Serum Albumin

To form the aqueous stabilized nanoparticle, the inverse nanocarrier, in some embodiments, needs to be coated. Example 2 detailed coating with a PEGylated amphiphilic block co-polymer. Here the aqueous stabilization using bovine serum albumin is described. To achieve this the well-established technique of Flash Nanoprecipitation (FNP)²⁰ was employed, which requires the dispersion of the nanocarrier in a water miscible organic solvent (such as 1,4-dioxane). As the formation of the initial inverse nanocarrier results in nanoparticles dispersed in chloroform along with excess spermine and triethylamine, an aqueous extraction with 150 mM sodium chloride was first performed to remove the residual 1,4-dioxane, spermine and triethylamine. Secondly, a solvent swap was performed with a rotary-evaporator to disperse the nanoparticles in 1,4-dioxane.

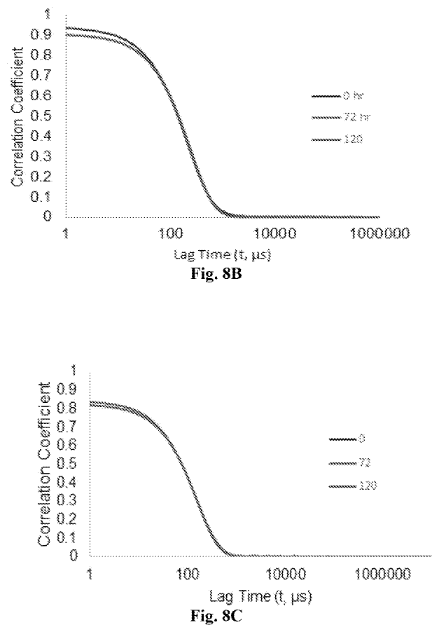

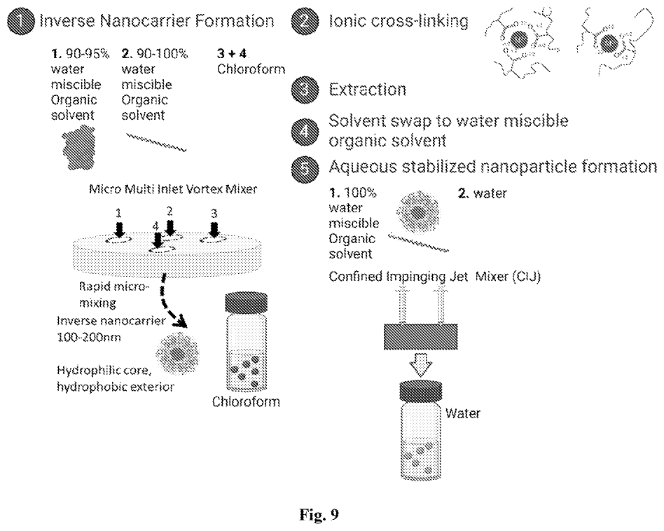

To coat the inverse nanocarrier with BSA, the inverse nanocarrier was dissolved in 1,4-dioxane. This nanocarrier was rapidly mixed with an antisolvent, water containing excess native or denatured BSA (1.5-2-fold mass excess), in a confined impinging jet mixer (CIJ) to directly generate BSA-coated DNase 1 nanoparticles, FIG. 8A. The stability of the BSA stabilized nanoparticles was assessed by incubating the at pH 5 at 4° C. and 37° C. over 120 h, FIGS. 8B and 8C.

### Example 4

### Encapsulation Efficiency and Loading

The aqueous extraction step in the processing of the inverse nanocarrier allows for the extraction of unencapsulated DNase 1 to be solubilized in the aqueous phase. Because of this it is possible to quantify encapsulation efficiency for the inverse nanocarrier by measuring DNase 1 content in the aqueous phase. Control experiments were performed where DNase 1 was mixed in the MIVM in the absence of stabilizing BCP, such that no nanoparticles were formed. Thus, it was confirmed that DNase 1 could be quantitatively extracted into the aqueous phase. Overall, two protein quantification methods (micro-BCA and protein absorbance at 280 nm) were employed to quantify that the encapsulation efficiency was 94.2±1.1%, and 96.4±5.5%, respectively. These results are summarized in Table 5. Combining the micro-BCA results with measurements by thermogravimetric analysis, it was possible to measure the loading capacity of the inverse nanocarrier. This was determined to be 3.68±0.28%. Thermogravimetric analysis also allowed for confirmation that there was no detectable loss of nanoparticle mass during the extraction step, as masses stayed the same before and post extraction (data not shown). This result, combined with the stability in the size and polydispersity of the particle through the processing steps, support that DNase 1 is encapsulated at the inverse nanocarrier stage was carried through to the PEG-coated nanoparticle.

TABLE 5
Encapsulation Efficiency and Loading
Capacity of the Inverse Nanocarrier

| Encapsulation | |
|---|---|
| Efficiency | Loading Capacity |
| 96.4 ± 5.5^{a} | 3.68 ± 0.28%^{c} |
| 94.2 ± 1.1^{b} | |

Footnotes:
^{a}Determined by protein absorbance measurements of the extract at A280 nm (N = 3 separate formulations, ±standard deviation).
^{b}Determined by micro-BCA assay of the extract (N = 3 separate formulations, ±standard deviation).
^{c}Quantified by thermogravimetric analysis (TGA) of the inverse nanocarrier and micro-BCA assay (N = 3-4 separate formulations, mean ± standard deviation).

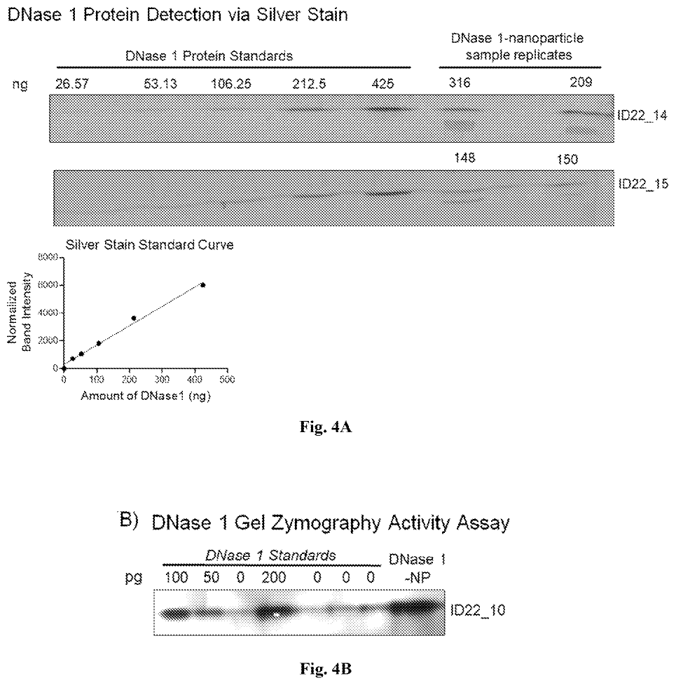

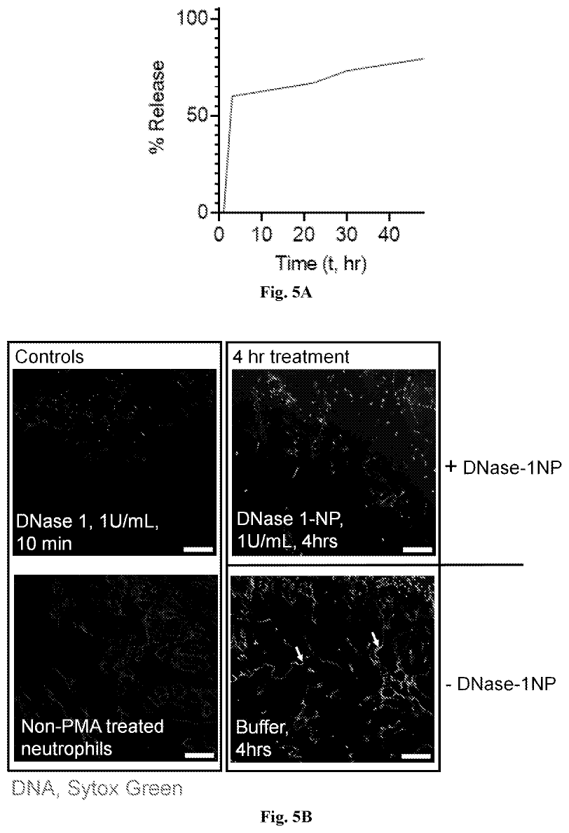

Aqueous stabilized nanoparticles were initially dialyzed against water using 300 kDa molecular weight cut-off dialysis tubing. For application in cell experiments they would then be sterile filtered with a 0.22 μm filter. It was confirmed that these final, purified, preparations contained DNase 1 protein by performing gel electrophoresis with aqueous stabilized nanoparticles, and subsequently staining the gel with the highly sensitive protein stain, Silver Stain (FIG. 4A). The Silver Stain process allows for the quantitation of DNase 1 down to about 20 ng. This method allows for an accurate quantitation of DNase 1 content in any given DNase1-nanoparticle aqueous solution for further experiments. As another orthogonal method to show that DNase 1 protein is present in the final preparation of aqueous stabilized DNase 1-nanoparticles, a method for measuring recovered DNase 1 activity utilizing denaturing polyacrylamide gel electrophoresis zymography was employed. Briefly, the nanoparticles were degraded by incubating them with 0.1M NaOH, and 5% SDS over 48 hours, and then loaded into a DNA-containing SDS-gel. Following protein refolding, we showed that DNase 1 activity could be recovered from aqueous stabilized DNase 1-nanoparticle samples (FIG. 4B). This method is not truly quantitative as band intensity poorly correlates with DNase 1 activity.

### Example 5

### DNase 1-Nanoparticles Degrade Neutrophil Extracellular Traps Ex Vivo

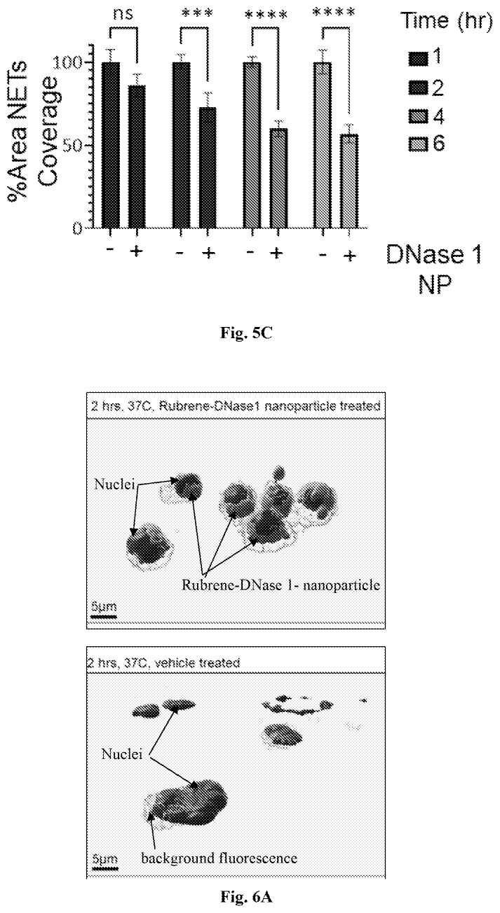

The purpose of the DNase-1 nanoparticles is to be delivered to venous clots where they release DNase 1 extra-cellularly to degrade the Neutrophil Extracellular Traps that help to hold clots together. Hence, it is important to assess the release profile of the nanoparticles. The drug release assayed by protein content into the release buffer, show a burst release in the first 3 hours, followed by sustained slow release for 48 h (FIG. 5A). To assess the ability of the nanoparticles to release DNAse with the ability to degrade extracellular DNS, NETs were generated ex vivo from bone-marrow derived murine neutrophils, stimulated with PMA in 96 well plates. The results of degradation of Neutrophil Extracellular Traps (NETs) by DNase 1 nanoparticles is shown in FIGS. 5B-5C. FIG. 5B includes representative fluorescence images of Sytox-green stained NETs. NETs were generated by stimulating murine bone marrow derived neutrophils with 100 nM PMA for 4 hours at 37° C. with 5% CO2 and humidity. These NETs were then incubated with 10 mM Tris-HCl buffer containing 3 mM CaCl₂) and 3 mM MgCl₂ for up to 6 hrs in the absence or presence of DNase 1 alone (1 U/mL) or DNase 1-nanoparticles (1 U/mL). After stopping the reaction with 2 mM EDTA and fixing the NETs with 2% PFA/1×PBS, the DNA was stained with Sytox Green and imaged fluorescently. Left-most upper panel: NETs degraded by DNase 1 alone (1 U/mL, 10 min). Left-most lower panel: Sytox-green staining produced by un-stimulated neutrophils. Middle upper and lower panel: NETs incubated for up to 6 hrs in the presence (upper) or absence (lower) of DNase1-NPs (1 U/mL). Right-most upper and lower panel: NETs incubated for up to 4 hrs in the presence (upper) or absence (lower) of DNase1-NPs (1 U/mL). FIG. 5C shows the quantification of NETs by Area coverage of NETs following treatment with DNase 1 alone or DNase 1 nanoparticles. 3-4 independent biological replicates are shown with 4-8 technical replicates per experiment. Time frames shown are 1 hr, 2 hr, 4 hr and 6 hr, from left to right, respectively, in FIG. 5C. Overall, these results show that DNase 1-nanoparticles can achieve NET degradation within 1 hr.

### Example 6

### Murine Neutrophils Rapidly Internalize DNase1-Nanoparticles

DNase 1-nanoparticles may be delivered to venous clots by ex vivo loading of autologous cells, which can then be adoptively transferred into a recipient. Alternatively, they may be injected intravenously and decorated with ligands that seek out infiltrating neutrophils. These data show that fluorescent DNase 1 nanoparticles are internalized by neutrophils via confocal microscopy. The presently disclosed data demonstrates that neutrophils internalize the fluorescent DNAse 1 nanoparticles with a plateau after about 1.5 hrs. Furthermore, it is demonstrated herein that viability of the neutrophils is maintained throughout the process, indicating that the DNase1 nanoparticles are not toxic at this concentration. Secondly, it is also confirmed that the signal observed with the fluorescent DNAse 1 nanoparticles is internalization by neutrophils, by comparing the flow cytometric median fluorescence intensity at low temperatures, where active processes such as internalization are inhibited. It was also found that internalization of the DNAse 1 nanoparticles does not obstruct important physiological functions of neutrophils such as the respiratory burst of activated neutrophils as shown by the reactive oxygen species production assay (FIG. 6D).

Turning to the Figures, Rubrene-DNase 1 nanoparticles were incubated for up to 1.5 hr with murine bone marrow derived neutrophils with a final concentration of about 0.2 μg/mL of rubrene at either 4° C. or 37° C. Neutrophils were then labelled with live/dead violet stain and CD45 antibody. FIG. 6A shows confocal fluorescence microscopy images of murine neutrophils incubated for 2 hrs at 37° C. either in the absence (bottom panel) or presence of rubrene-DNase 1 nanoparticles (top panel) at a concentration of about 0.4 μg/mL of rubrene. FIG. 6B shows quantification of % positive cells for rubrene-DNase 1 nanoparticles over 1.5 hrs. FIG. 6C shows viability of neutrophils over 1.5 hr incubation at either 4° C. or 37° C. Geometric mean fluorescence intensity of neutrophils incubated with rubrene DNase 1 nanoparticles. Data represents N=3-4 biological replicates with N=1-2 technical replicates. Data is presented as the mean±S.E.M. % Cells positive were analyzed via multiple t-test comparison of row-by-row. ***=P<0.001, ****=P<0.0001. FIG. 6D shows effect of internalization of DNase1-nanoparticles on neutrophil stimulation (unstimulated, left two bars; PMA (100 nM), middle two bars; PMA (100 nM), Ionomycin (1 uM), right two bars).

### Example 7

### Exogenous Murine Neutrophils Loaded with DNase1-Nanoparticles Enter Pre-Formed Clots

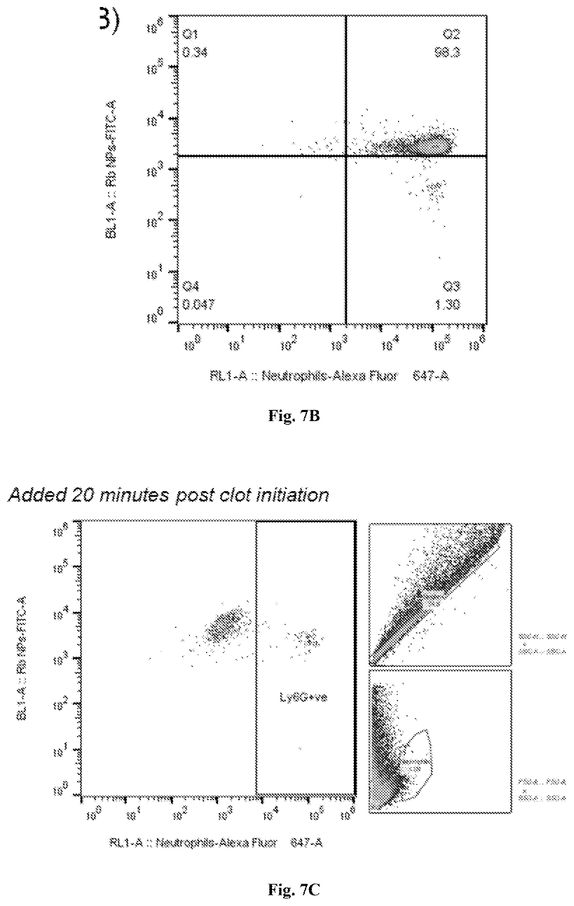

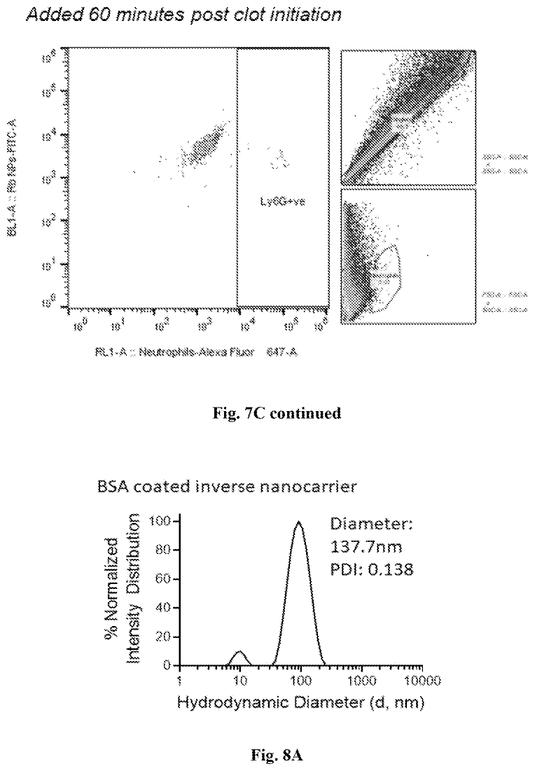

DNase 1-nanoparticles may be delivered to venous clots by ex vivo loading of autologous cells, which can then be adoptively transferred into a recipient. In this study isolated neutrophils were loaded as described in Example 6 and labeled with fluorescent anti Ly6G-AF647 conjugated antibody. Clots were formed ex vivo by adding tissue factor and platelet activating factor to recalcified murine blood. Neutrophils loaded with DNAse1 nanoparticles were added at 20 or 60 minutes after starting the clotting process. Time was allowed for the neutrophils to migrate into the clot by incubating overnight at 37° C. The clot was then prepared as a single cell suspension for flow cytometry. As shown in FIGS. 7A-7C, exogenous neutrophils loaded with DNAse 1 nanoparticles are found in the pre-formed clot.

FIG. 7A shows a schematic overview of experimental protocol. Briefly, murine bone marrow derived neutrophils were incubated with rubrene containing DNase1-NPs (˜0.4 μg/mL of rubrene) for 2 hrs at 37° C. Following this, murine venous blood was isolated and an in vitro blood clot was formed via recalcification and treatment with Tissue Factor and Platelet Activating Factor. 20 minutes or 60 minutes following clot formation, exogenous DNAse1-NP loaded neutrophils were added to the blood clot and incubated overnight. The clot was then retrieved and prepared as a fixed, individual cell suspension for analysis by flow cytometry. FIG. 7B shows that 98% of isolated Ly6G-AF647 labelled murine bone marrow derived neutrophils were positive for rubrene-containing DNase-1 NPs following 2 hr incubation. Isolated neutrophils were utilized to gate the neutrophil population in SSC-A vs FSC-A. FIG. 7C confirms that exogenous neutrophil populations were detected in blood clot single cell suspensions. The single cell suspension was gated to cut off cell debris, then eliminate doublets and aggregates, and then neutrophils as determined by the previously isolated population in FIG. 7B, and then finally the positive population of Ly6G stained neutrophils was selected.

### Example 8

### Conclusions from Examples 1-7

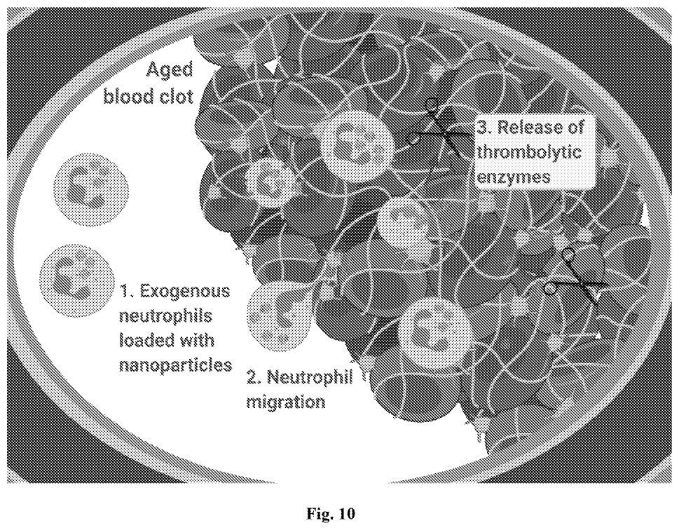

Disclosed herein is a protocol utilizing iFNP with a unique solvent system and ions that act to preserve protein activity, to generate a polymeric nanoparticle encapsulating the therapeutic protein DNase 1. The data herein demonstrate that DNase 1 can be effectively encapsulated with an encapsulation efficiency of 94.2±1.1%, and 96.4±5.5%, as measured by either micro-BCA or protein absorbance at 280 nm, respectively. Additionally, it was found that for the inverse nanocarrier the loading capacity was 3.68±0.28%. The presence of DNase 1 in aqueous stabilized DNase-1 nanoparticles was confirmed by both by SDS-gel electrophoresis with Silver Stain protein detection and gel zymography measurements. Additionally, it was shown that DNase 1 can then be released to degrade NETs. It was also shown that the nanoparticles can be stabilized directly with BSA without the need of a second layer of block co-polymer. Additionally, the data show that murine bone marrow derived neutrophils can internalize these DNase 1-nanoparticles, while maintaining their viability and without inducing neutrophil activation. Furthermore, the data show that exogenous neutrophils loaded with the DNAse 1 nanoparticles can migrate inside a pre-formed clot. Based on the research disclosed herein, the disclosed formulations and therapeutics, particularly those with DNase 1-nanoparticles, have the ability to degrade NETs in blood clots and that neutrophil-mediated delivery of nanoparticles in vivo venous thrombi is achievable (see the schematic illustration in FIG. 10).

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. Prevention, C. f. D. C. a., Data and Statistics on Venous Thromboembolism. 2019.
2. Kahn, S. R., The post-thrombotic syndrome: progress and pitfalls. Br J Haematol 2006, 134 (4), 357-65.
3. Suh, Y. J.; Hong, H.; Ohana, M.; Bompard, F.; Revel, M. P.; Valle, C.; Gervaise, A.; Poissy, J.; Susen, S.; Hékimian, G.; Artifoni, M.; Periard, D.; Contou, D.; Delaloye, J.; Sanchez, B.; Fang, C.; Garzillo, G.; Robbie, H.; Yoon, S. H., Pulmonary Embolism and Deep Vein Thrombosis in COVID-19: A Systematic Review and Meta-Analysis. Radiology 2021, 298 (2), E70-e80.
4. Czaplicki, C.; Albadawi, H.; Partovi, S.; Gandhi, R. T.; Quencer, K.; Deipolyi, A. R.; Oklu, R., Can thrombus age guide thrombolytic therapy?Cardiovascular Diagnosis and Therapy 2017, 7, S186-S196.
5. Kim, Y. D.; Nam, H. S.; Kim, S. H.; Kim, E. Y.; Song, D.; Kwon, I.; Yang, S. H.; Lee, K.; Yoo, J.; Lee, H. S.; Heo, J. H., Time-Dependent Thrombus Resolution after Tissue-Type Plasminogen Activator in Patients with Stroke and Mice. Stroke 2015, 46(7), 1877-1882.
6. Savchenko, A. S.; Martinod, K.; Seidman, M. A.; Wong, S. L.; Borissoff, J. I.; Piazza, G.; Libby, P.; Goldhaber, S. Z.; Mitchell, R. N.; Wagner, D. D., Neutrophil extracellular traps form predominantly during the organizing stage of human venous thromboembolism development. Journal of Thrombosis and Haemostasis 2014, 12 (6), 860-870.
7. Thålin, C.; Hisada, Y.; Lundström, S.; Mackman, N.; Wallén, H., Neutrophil Extracellular Traps. Arteriosclerosis, thrombosis, and vascular biology 2019, 39 (9), 1724-1738.
8. Fuchs, T. A.; Brill, A.; Duerschmied, D.; Schatzberg, D.; Monestier, M., Myers Jr, D. D.; Wrobleski, S. K.; Wakefield, T. W.; Hartwig, J. H.; Wagner, D. D., Extracellular DNA traps promote thrombosis. Proceedings of the National Academy of Sciences of the United States of America 2010, 107 (36), 15880-15885.
9. Martinod, K.; Wagner, D. D., Thrombosis: Tangled up in NETs. Blood 2014, 123 (18), 2768-2776.
10. Novotny, J.; Chandraratne, S.; Weinberger, T.; Philippi, V.; Stark, K.; Ehrlich, A.; Pircher, J.; Konrad, I.; Oberdieck, P.; Titova, A.; Hoti, Q.; Schubert, I.; Legate, K. R.; Urtz, N.; Lorenz, M.; Pelisek, J.; Massberg, S.; von Briihl, M. L.; Schulz, C., Histological comparison of arterial thrombi in mice and men and the influence of Cl-amidine on thrombus formation. PLoS ONE 2018, 13 (1).
11. Brill, A.; Fuchs, T. A.; Savchenko. A. S.; Thomas, G. M.; Martinod, K.; de Meyer, S. F.; Bhandari, A. A.; Wagner, D. D., Neutrophil extracellular traps promote deep vein thrombosis in mice. Journal of Thrombosis and Haemostasis 2012, 10 (1), 136-144.
12. Semeraro, F.; Ammollo, C. T.; Morrissey, J. H.; Dale, G. L.; Friese, P.; Esmon, N. L.; Esmon, C. T., Extracellular histones promote thrombin generation through platelet-dependent mechanisms: Involvement of platelet TLR2 and TLR4. Blood 2011,118 (7), 1952-1961.
13. Jansen, M. P. B.; Emal, D.; Teske, G. J. D.; Dessing, M. C.; Florquin, S.; Roelofs, J. J. T. H., Release of extracellular DNA influences renal ischemia reperfusion injury by platelet activation and formation of neutrophil extracellular traps. Kidney International 2016.
14. Varjú, I.; Kolev, K., Networks that stop the flow: A fresh look at fibrin and neutrophil extracellular traps. Thrombosis Research 2019, 182, 1-11.
15. Longstaff, C.; Varjú, I.; Sótonyi, P.; Szabó, L.; Krumrey, M.; Hoell, A.; Bóta, A.; Varga, Z.; Komorowicz, E.; Kolev, K., Mechanical stability and fibrinolytic resistance of clots containing fibrin, DNA, and histones. Journal of Biological Chemistry 2013, 288 (10), 6946-6956.
16. Gould, T. J.; Vu, T. T.; Stafford, A. R.; Dwivedi, D. J.; Kim, P. Y.; Fox-Robichaud, A. E.; Weitz, J. I.; Liaw, P. C., Cell-Free DNA Modulates Clot Structure and Impairs Fibrinolysis in Sepsis. Arteriosclerosis, thrombosis, and vascular biology 2015, 35 (12), 2544-2553.
17. Levit, S. L.; Yang, H.; Tang, C., Rapid Self-Assembly of Polymer Nanoparticles for Synergistic Codelivery of Paclitaxel and Lapatinib via Flash NanoPrecipitation. Nanomaterials (Basel) 2020, 10 (3).
18. Tao, J.; Chow, S. F.; Zheng, Y., Application of flash nanoprecipitation to fabricate poorly water-soluble drug nanoparticles. Acta Pharm Sin B 2019, 9 (1), 4-18.
19. Markwalter, C. E.; Pagels, R. F.; Hejazi, A. N.; Gordon, A. G. R.; Thompson, A. L.; Prud'homme, R. K., Polymeric Nanocarrier Formulations of Biologics Using Inverse Flash NanoPrecipitation. Aaps j 2020, 22 (2), 18.
20. Markwalter, C. E.; Pagels, R. F.; Wilson, B. K.; Ristroph, K. D.; Prud'homme, R. K., Flash NanoPrecipitation for the Encapsulation of Hydrophobic and Hydrophilic Compounds in Polymeric Nanoparticles. J Vis Exp 2019, (143).
21. Jiménez-Alcázar, M.; Rangaswamy, C.; Panda, R.; Bitterling, J.; Simsek, Y. J.; Long, A. T.; Bilyy, R.; Krenn, V.; Renné, C.; Renné, T.; Kluge, S.; Panzer, U.; Mizuta, R.; Mannherz, H. G.; Kitamura, D.; Herrmann, M.; Napirei, M.; Fuchs, T. A., Host DNases prevent vascular occlusion by neutrophil extracellular traps. Science 2017, 358 (6367), 1202-1206.
22. Rosenthal, A. L.; Lacks, S. A., Nuclease detection in SDS-polyacrylamide gel electrophoresis. Anal Biochem 1977, 80 (1), 76-90.
23. Bisso, P. W.; Gaglione, S.; Guimaraes, P. P. G.; Mitchell, M. J.; Langer, R., Nanomaterial Interactions with Human Neutrophils. ACS Biomaterials Science and Engineering 2018, 4 (12), 4255-4265.
24. Hakkim, A.; Fürmrohr, B. G.; Amann, K.; Laube, B.; Abed, U. A.; Brinkmann, V.; Herrmann, M.; Voll, R. E.; Zychlinsky, A., Impairment of neutrophil extracellular trap degradation is associated with lupus nephritis. Proc Natl Acad Sci USA 2010, 107 (21), 9813-8.
25. Feng, J.; Markwalter, C. E.; Tian, C.; Armstrong, M.; Prud'homme, R. K., Translational formulation of nanoparticle therapeutics from laboratory discovery to clinical scale. J Transl Med 2019, 17 (1), 200.
26. Mattos, C.; Ringe, D., Proteins in organic solvents. Curr Opin Struct Biol 2001, 11 (6), 761-4.
27. Chen, W. J.; Liao, T. H., Structure and function of bovine pancreatic deoxyribonuclease I. Protein Pept Lett 2006, 13 (5), 447-53.
28. Markwalter, C. E.; Pagels, R. F.; Hejazi, A. N.; Gordon, A. G. R.; Thompson, A. L.; Prud'homme, R. K., Polymeric Nanocarrier Formulations of Biologics Using Inverse Flash NanoPrecipitation. AAPS Journal 2020, 22 (2).
29. Jiménez-Alcázar, M.; Rangaswamy, C.; Panda, R.; Bitterling, J.; Simsek, Y. J.; Long, A. T.; Bilyy, R.; Krenn, V.; Renné, C.; Renné, T.; Kluge, S.; Panzer, U.; Mizuta, R.; Mannherz, H. G.; Kitamura, D.; Herrmann, M.; Napirei, M.; Fuchs, T. A., Host DNases prevent vascular occlusion by neutrophil extracellular traps. Science 2017, 358 (6367), 1202-1206.
30. Hakkim, A., Fürnrohr, B. G.; Amann, K.; Laube, B.; Abed, U. A.; Brinkmann, V.; Herrmann, M.; Voll, R. E.; Zychlinsky, A., Impairment of neutrophil extracellular trap degradation is associated with lupus nephritis. Proc Natl Acad Sci USA 2010, 107 (21), 9813-8.
31. Ban, E.; Kim, A., Coacervates: Recent developments as nanostructure delivery platforms for therapeutic biomolecules. International Journal of Pharmaceutics 2022, 122058.
32. Barthold, S.; Kletting, S.; Taffner, J.; de Souza Carvalho-Wodarz, C.; Lepeltier, E.; Loretz, B.; Lehr, C.-M., Preparation of nanosized coacervates of positive and negative starch derivatives intended for pulmonary delivery of proteins. Journal of Materials Chemistry B 2016, 4 (13), 2377-2386.
33. Bilati, U.; Allémann, E.; Doelker, E., Poly (D, L-lactide-co-glycolide) protein-loaded nanoparticles prepared by the double emulsion method—processing and formulation issues for enhanced entrapment efficiency. Journal of microencapsulation 2005, 22 (2), 205-214.
34. Lopes, M. A.; Abrahim-Vieira, B.; Oliveira, C.; Fonte, P.; Souza, A. M.; Lira, T.; Sequeira, J. A.; Rodrigues, C. R.; Cabral, L. M.; Sarmento, B., Probing insulin bioactivity in oral nanoparticles produced by ultrasonication-assisted emulsification/internal gelation. International Journal of Nanomedicine 2015, 5865-5880.
35. Lamprecht, A.; Ubrich, N.; Pérez, M. H.; Lehr, C.-M.; Hoffman, M.; Maincent, P., Biodegradable monodispersed nanoparticles prepared by pressure homogenization-emulsification. International journal of pharmaceutics 1999, 184 (1), 97-105.
36. Huang, J.; Shu, Q.; Wang, L.; Wu, H.; Wang, A. Y.; Mao, H., Layer-by-layer assembled milk protein coated magnetic nanoparticle enabled oral drug delivery with high stability in stomach and enzyme-responsive release in small intestine. Biomaterials 2015, 39, 105-113.
37. Gu, X.; Wang, J.; Wang, Y.; Wang, Y.; Gao, H.; Wu, G., Layer-by-layer assembled polyaspartamide nanocapsules for pH-responsive protein delivery. Colloids and Surfaces B: Biointerfaces 2013, 108, 205-211.
38. Tan, Y. F.; Mundargi, R. C.; Chen, M. H. A.; Lessig, J.; Neu, B.; Venkatraman, S. S.; Wong, T. T., Layer-by-layer nanoparticles as an efficient siRNA delivery vehicle for SPARC silencing. Small 2014, 10 (9), 1790-1798.
39. Marante, T.; Viegas, C.; Duarte, I.; Macedo, A. S.; Fonte, P., An overview on spray-drying of protein-loaded polymeric nanoparticles for dry powder inhalation. Pharmaceutics 2020, 12 (11), 1032.
40. Schiller, S.; Hanefeld, A.; Schneider, M.; Lehr, C.-M., Towards a continuous manufacturing process of protein-loaded polymeric nanoparticle powders. AAPS PharmSciTech 2020, 21, 1-5.
41. Erdinc, B.; Neufeld, R., Protein micro and nanoencapsulation within glycol-chitosan/Ca2+/alginate matrix by spray drying. Drug development and industrial pharmacy 2011, 37 (6), 619-627.
42. Vishali, D.; Monisha, J.; Sivakamasundari, S.; Moses, J.; Anandharamakrishnan, C., Spray freeze drying: Emerging applications in drug delivery. Journal of Controlled Release 2019, 300, 93-101.
43. Schiffter, H.; Condliffe, J.; Vonhoff, S., Spray-freeze-drying of nanosuspensions: the manufacture of insulin particles for needle-free ballistic powder delivery. Journal of the Royal Society Interface 2010, 7 (suppl_4), S483-S500.
44. Leach, W. T.; Simpson, D. T.; Val, T. N.; Anuta, E. C.; Yu, Z.; Williams III, R. O.; Johnston, K. P., Uniform encapsulation of stable protein nanoparticles produced by spray freezing for the reduction of burst release. Journal of pharmaceutical sciences 2005, 94 (1), 56-69.
45. Tenchov, R.; Bird, R.; Curtze, A. E.; Zhou, Q., Lipid nanoparticles—from liposomes to mRNA vaccine delivery, a landscape of research diversity and advancement. ACS nano 2021, 15 (11), 16982-17015.
46. Bi, D.; Unthan, D. M.; Hu, L.; Bussmann, J.; Remaut, K.; Barz, M.; Zhang, H., Polysarcosine-based lipid formulations for intracranial delivery of mRNA. Journal of Controlled Release 2023, 356, 1-13.
47. Yang, R.; Gao, R.; Li, F.; He, H.; Tang, X., The influence of lipid characteristics on the formation, in vitro release, and in vivo absorption of protein-loaded SLN prepared by the double emulsion process. Drug development and industrial pharmacy 2011, 37 (2), 139-148.
48. van Swaay, D.; DeMello, A., Microfluidic methods for forming liposomes. Lab on a Chip 2013, 13 (5), 752-767.
49. Warne, N.; Ruesch, M.; Siwik, P.; Mensah, P.; Ludwig, J.; Hripcsak, M.; Godavarti, R.; Prigodich, A.; Dolsten, M., Delivering 3 billion doses of Comimaty in 2021. Nature Biotechnology 2023, 41 (2), 183-188.
50. Zheng, H.; Tao, H.; Wan, J.; Lee, K. Y.; Zheng, Z.; Leung, S. S. Y., Preparation of drug-loaded liposomes with multi-inlet vortex mixers. Pharmaceutics 2022, 14 (6), 1223.
51. Chang, W.-K.; Tai, Y.-J.; Chiang, C.-H.; Hu, C.-S.; Hong, P.-D.; Yeh, M.-K., The comparison of protein-entrapped liposomes and lipoparticles: preparation, characterization, and efficacy of cellular uptake. International journal of nanomedicine 2011, 2403-2417.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A therapeutic composition for treating a subject, the therapeutic composition comprising:
a nanoparticle;
an active agent, wherein the active agent is encapsulated within the nanoparticle; and
a delivery vehicle for targeted delivery of the nanoparticle and encapsulated agent to a target tissue in the subject.

2. The therapeutic composition of claim 1, wherein the active agent is releasable from the delivery vehicle at the target tissue.

3. The therapeutic composition of any of claims 1 or 2, wherein the active agent comprises a small molecule, nucleic acid, therapeutic protein and/or an active enzyme.

4. The therapeutic composition of any of claims 1 to 3, wherein the active agent comprises Deoxyribonuclease 1 (DNase 1), optionally wherein the active agent comprises a nucleic acid encoding for DNase 1.

5. The therapeutic composition of claim 1, wherein the delivery vehicle is a functionalized delivery vehicle, wherein the functionalized delivery vehicle comprises a cell or cellular component for cell-mediated delivery of the nanoparticle.

6. The therapeutic composition of claim 5, wherein the cell or cellular component for cell-mediated delivery of the nanoparticle comprises an immune cell.

7. The therapeutic composition of claim 6, wherein the cell or cellular component for cell-mediated delivery of the nanoparticle comprises a macrophage or neutrophil.

8. The therapeutic composition of claim 6, wherein the cell or cellular component for cell-mediated delivery of the nanoparticle comprises any autologous cell.

9. The therapeutic composition of any of claims 5 to 8, wherein the functionalized delivery vehicle for targeted delivery of the nanoparticle to a target tissue in the subject comprises a delivery vehicle decorated with ligands with an affinity for immune cells.

10. The therapeutic composition of claim 9, wherein the functionalized delivery vehicle for targeted delivery of the nanoparticle to a target tissue in the subject comprises a delivery vehicle decorated with ligands with an affinity for a macrophage or neutrophil, optionally any autologous cell.

11. The therapeutic composition of any of claims 1 to 10, wherein the active agent maintains activity upon release from the delivery vehicle at the target tissue.

12. The therapeutic composition of claim 11, wherein the active agent maintains activity of at least about 50%, about 60%, about 70%, about 80%, about 90%, about 95%, about 99% or more of an activity of the active agent prior to release from the delivery vehicle.

13. The therapeutic composition of claim 11, wherein the active agent comprises an enzyme with maintained enzymatic activity upon release at the target tissue.

14. The therapeutic composition of claim 13, wherein the enzyme maintains activity of at least about 50%, about 60%, about 70%, about 80%, about 90%, about 95%, about 99% or more of an activity of the enzyme prior to release from the delivery vehicle.

15. The therapeutic composition of any of claims 1 to 14, wherein the nanoparticle is a polymeric nanoparticle, wherein the nanoparticle is surrounded by a protective layer of polymers.

16. The therapeutic composition of claim 15, wherein the protective layer of polymers comprises a plurality of layers of polymers, optionally at least one, two or three layers of polymers.

17. The therapeutic composition of claim 16, wherein at least one of the plurality of layers of polymers is substituted for a layer of protein.

18. The therapeutic composition of claim 17, wherein the layer of protein comprises albumin.

19. The therapeutic composition of claim 16 or 17, wherein the layer of protein stabilizes the nanoparticle.

20. The therapeutic composition of any of claims 15 to 19, wherein the protective layer of polymers comprises one or more layers of polyethylene glycol (PEG).

21. The therapeutic composition of any of claims 15 to 20, wherein nanoparticle comprises a poly-acrylic acid, complexed with spermine and/or triethylamine, and coated one or more layers of PEG.

22. The therapeutic composition of any of claims 1 to 21, wherein the nanoparticle comprises dual encapsulated active agents, optionally two separate active agents comprising a biologic and a small molecule.

23. The therapeutic composition of any of claims 1 to 22, wherein the polymeric nanoparticle for encapsulation of an active agent is made by inverse Flash Nanopreciptation (iFNP) with stabilization of the nanoparticle using a polymer, an aqueous protein, or a combination thereof.

24. The therapeutic composition of any of claims 1 to 23, further comprising CaCl2) to aid in the maintance of DNAse 1 activity upon release.

25. A therapeutic composition for targeting the destruction of a neutrophil extracellular trap (NET) in a venous thrombus in a subject, the therapeutic composition comprising a nanoparticle encapsulating a Deoxyribonuclease 1 (DNase 1) enzyme, wherein the DNase 1 is releasable from the nanoparticle at the venous thrombus, wherein the DNase 1 maintains activity upon release.

26. The therapeutic composition of claim 25, further comprising CaCl₂) to aid in the maintenance of DNAse 1 activity upon release.

27. The therapeutic composition of claims 25 or 26, wherein the nanoparticle comprises a layer of polyethylene glycol (PEG) and/or a layer of albumin.

28. The therapeutic composition of any of claims 25 to 27, further comprising a cell or cellular component for cell-mediated delivery of the nanoparticle to a venous thrombus in the subject.

29. The therapeutic composition of claim 28, wherein the cell or cellular component for cell-mediated delivery of the nanoparticle comprises an immune cell.

30. The therapeutic composition of claim 28, wherein the cell or cellular component for cell-mediated delivery of the nanoparticle comprises a macrophage or neutrophil.

31. The therapeutic composition of claim 28, wherein the cell or cellular component for cell-mediated delivery of the nanoparticle comprises any autologous cell.

32. The therapeutic composition of any of claims 25 to 27, wherein the functionalized delivery vehicle for targeted delivery of the nanoparticle to a target tissue in the subject comprises a delivery vehicle decorated with ligands with an affinity for immune cells.

33. The therapeutic composition of claim 32, wherein the functionalized delivery vehicle for targeted delivery of the nanoparticle to a target tissue in the subject comprises a delivery vehicle decorated with ligands with an affinity for a macrophage or neutrophil, optionally any autologous cell.

34. A method of treating a condition in a subject in need of treatment, the method comprising administering to a subject a therapeutic composition of any of claims 1 to 33, whereby the subject's condition is treated.

35. The method of claim 34, wherein the subject is suffering from an inflammatory condition, wherein the inflammatory condition recruits neutrophils and/or macrophages, whereby administration of the therapeutic composition comprising a functionalized delivery vehicle or cell carrier targets the therapeutic composition to the site of the inflammatory condition.

36. The method of any of claims 34 to 35, wherein the subject is suffering from venous thrombus, deep vein thrombosis, or a related blood clotting disease and/or condition.

37. The method of any of claims 34 to 36, wherein administration of the therapeutic composition targets destruction of a neutrophil extracellular trap (NET) in a venous thrombus of the subject, optionally wherein intra-thrombus delivery of a thrombolytic enzyme, e.g. DNase 1, confers thrombolysis of venous thrombi in the subject without impacting surrounding tissues.

38. The method of any of claims 34 to 37, wherein the therapeutic composition confers intra-thrombus delivery of a thrombolytic enzyme to a hardened thrombus with greater penetration of the thrombus as compared to other treatments, whereby the therapeutic composition confers greater thrombolysis of a thrombus with fewer side-effects.

39. A method of making a polymeric nanoparticle for encapsulation of an active agent, the method comprising inverse Flash Nanopreciptation (iFNP) with stabilization of the nanoparticle with a polymer, an aqueous protein instead of a polymer, or a combination thereof.

40. The method of claim 39, further comprising using dioxane, e.g. 1,4-dioxane, and/or CaCl₂), to maintain activity of the active agent.
